(19) United States
(12) Patent Application Publication
(10) Pub. No.: US 2025/0281051 A1
(43) Pub. Date: Sep. 11, 2025

(54) DISPLAY DEVICE FOR BIOMETRIC DETECTION

(71) Applicant: SAMSUNG DISPLAY CO., LTD., YONGIN-SI (KR)

(72) Inventor: Chul KIM, Yongin-si (KR)

(21) Appl. No.: 18/907,632

(22) Filed: Oct. 7, 2024

(30) Foreign Application Priority Data
Mar. 8, 2024 (KR) 10-2024-0032962

(51) Int. Cl. A61B 5/021; A61B 5/00

(52) U.S. Cl. CPC A61B 5/02141; A61B 5/02108; A61B 5/742

(57) ABSTRACT

A display device includes a display panel including display pixels and light sensing pixels arranged in a display area of the display panel, a display scan driver configured to drive the display pixels to emit light, a light sensing scan driver configured to drive the light sensing pixels to sense light, and a main driving circuit. The main driving circuit is configured to obtain, using a biometric information detection device, a first reference pulse wave signal, a second reference pulse wave signal, a first pulse wave signal, and a second pulse wave signal, generate a first reference correction value and a second reference correction value based on the first reference pulse wave signal and the second reference pulse wave signal, respectively, and generate biometric information based on the first pulse wave signal, the second pulse wave signal, the first reference correction value, and the second reference correction value.

### CROSS-REFERENCE TO RELATED APPLICATION

This U.S. non-provisional patent application claims priority under 35 U.S.C. § 119 to Korean Patent Application No. 10-2024-0032962, filed on Mar. 8, 2024, in the Korean Intellectual Property Office, the disclosure of which is incorporated by reference in its entirety.

### BACKGROUND

The present disclosure relates to a display device, and more specifically to a display device capable of accurately detecting and generating biometric information based on a plurality of pulse wave signals.

With the rise of information-oriented society, the demand on display devices for displaying images in various ways has increased. These display devices may be integrated into various electronic devices, such as a smart phone, a digital camera, a laptop computer, a tablet PC, a navigation system, and a smart television. In some cases, portable display devices such as smartphones, tablet PCs, etc., provide various functions such as image capturing, fingerprint recognition, face recognition, etc.

Recently, with the healthcare industry gaining attention, methods have been developed to obtain biometric information related to health more easily. In some cases, for example, some methods replace a traditional blood pressure measuring device using an oscillometric method with a portable blood pressure measuring device. However, the portable blood pressure measuring device requires a separate light source, sensor, and display. As a result, to execute the conventional methods require to separately carry the portable blood pressure measuring device in addition to the portable smartphone or tablet PC, thus introduces inconvenience. In some cases, efforts have been made to combine a portable display device such as a smart phone, a tablet PC, or the like with a portable blood pressure measuring device. However, methods for measuring various pieces of biometric information such as a heart rate, heart rate variability, respiration, a cardiovascular disease, oxygen saturation, etc. would require a separate portable device capable of measuring these biometric information.

### SUMMARY

A display device includes a display panel including display pixels and light sensing pixels arranged in a display area of the display panel, a display scan driver configured to drive the display pixels to emit light, a light sensing scan driver configured to drive the light sensing pixels to sense light, and a main driving circuit. In one aspect, the main driving circuit is configured to perform operations including obtaining, using a biometric information detection device, a first reference pulse wave signal, a second reference pulse wave signal, a first pulse wave signal, and a second pulse wave signal, generating a first reference correction value and a second reference correction value based on the first reference pulse wave signal and the second reference pulse wave signal, respectively, and generating biometric information based on the first pulse wave signal, the second pulse wave signal, the first reference correction value, and the second reference correction value.

A display device includes a display panel including display pixels and light sensing pixels arranged in a display area of the display panel, a display scan driver configured to drive the display pixels to emit light, a light sensing scan driver configured to drive the light sensing pixels to sense light, and a main driving circuit. In one aspect, the main driving circuit is configured to perform operations including obtaining, using a biometric information detection device, a first reference pulse wave signal, a second reference pulse wave, a first pulse wave signal, and a second pulse wave signal, generating a first reference correction value and a second reference correction value based on the first reference pulse wave signal and the second reference pulse wave signal, and generating biometric information based on the first pulse wave signal, the second pulse wave signal, the first reference correction value, and the second reference correction value. In one aspect, the main driving circuit guides a biometric signal detection process by displaying an application program screen, generating a biometric signal based on the first pulse wave signal and second pulse wave signal, generating a final measuring result based on the biometric signal, and displaying the final measuring result on the application program screen.

A method for generating biometric information using a display device, the method includes obtaining, using a biometric information detection device, a first reference pulse wave signal through a left arm, obtaining, using a display device, a first pulse wave signal through a right finger, generating a first reference correction value with respect to the right finger based on the first reference pulse wave signal and the first pulse wave signal, and generating biometric information based on the first reference correction value.

### BRIEF DESCRIPTION OF THE DRAWINGS

FIG. 1 is an example of a perspective view of a display device according to an embodiment of the present inventive concept.

FIG. 2 is an example of a plan view illustrating an arrangement structure of a display panel and a main driving circuit as shown in FIG. 1.

FIGS. 3 and 4 are examples of a side view illustrating the configuration of the display device as shown in FIG. 1.

FIG. 5 is an example of a schematic layout diagram of the display panel illustrated in FIGS. 1 to 4.

FIG. 6 is an example of a layout diagram illustrating a display area according to an embodiment of the present inventive concept.

FIG. 7 is an example of a circuit diagram illustrating a display pixel and a light sensing pixel according to an embodiment of the present inventive concept.

FIG. 8 is an example of a block diagram illustrating a biometric signal detection unit of a main driving circuit illustrated in FIGS. 1 to 3.

FIG. 9 is an example of a diagram illustrating a process for setting reference correction values based on a pulse wave signal detected from a right finger of a user.

FIG. 10 is an example of a waveform diagram illustrating a detection waveform of a pulse wave signal from a biometric signal measuring device of FIG. 9.

FIG. 11 is an example of a waveform diagram illustrating a detection waveform of a pulse wave signal from a display device of FIG. 9.

FIG. 12 is an example of a diagram illustrating a process for setting reference correction values based on a pulse wave signal detected from a left finger of a user.

FIG. 13 is an example of a waveform diagram illustrating a waveform detection of a pulse wave signal from a biometric signal measuring device of FIG. 12.

FIG. 14 is an example of a waveform diagram illustrating a waveform detection of a pulse wave signal from a display device of FIG. 12.

FIG. 15 is an example of a diagram illustrating a measuring process of a plurality of pulse wave signals and biometric information.

FIG. 16 is an example of a diagram illustrating a displayed image display screen when a pulse wave signal is detected during a measurement period.

FIG. 17 is an example of a waveform diagram illustrating pulse wave signals detected from different touch positions.

FIG. 18 is an example of a diagram illustrating a method for calculating blood pressure information using a machine learning algorithm according to an embodiment of the present inventive concept.

FIG. 19 is an example of a graph illustrating a method for calculating information on a heart rate and respiration among biometric information according to an embodiment of the present inventive concept.

FIG. 20 is an example of a graph illustrating a method for calculating information on blood vessel elasticity among biometric information according to an embodiment of the present inventive concept.

FIG. 21 is an example of a graph illustrating a method for calculating information on a cardiovascular disease among biometric information according to an embodiment of the present inventive concept.

FIG. 22 is an example of a graph illustrating a method for calculating information on oxygen saturation among biometric information according to an embodiment of the present inventive concept.

### DETAILED DESCRIPTION OF THE EMBODIMENTS

Hereinafter, the present disclosure is described in detail with reference to the accompanying drawings, in which preferred embodiments of the disclosure are shown. However, the disclosure may include different embodiments that might not be shown in the drawings, and thus should not be construed as limited to the embodiments set forth herein. Rather, these embodiments are provided to ensure the disclosure is thorough and complete, effectively conveying the scope of the disclosure to those skilled in the art.

It will also be understood that when a layer is referred to as being “on” or “under” another layer or substrate, it can be directly on the other layer or substrate, or intervening layers may also be present. For example, when the disclosure describes a first layer disposed on a second layer, then the first layer may be directly disposed on the second layer. In some cases, for example, a third layer may be disposed between the first layer and the second layer. In some aspects, the same reference numbers indicate the same components throughout the specification.

It will be understood that, although the terms “first,” “second,” etc. may be used herein to describe various elements, these elements should not be limited by these terms. These terms are only used to distinguish one element from another element. For example, a first element discussed below could be termed a second element without departing from the teachings and spirit of the present disclosure. Similarly, the second element could also be termed the first element.

Each of the features of the various embodiments of the present disclosure may be combined with each other, partially or fully, allowing for various technically interlocking and driving possibilities. Each embodiment may be implemented independently of each other or may be implemented together in an association.

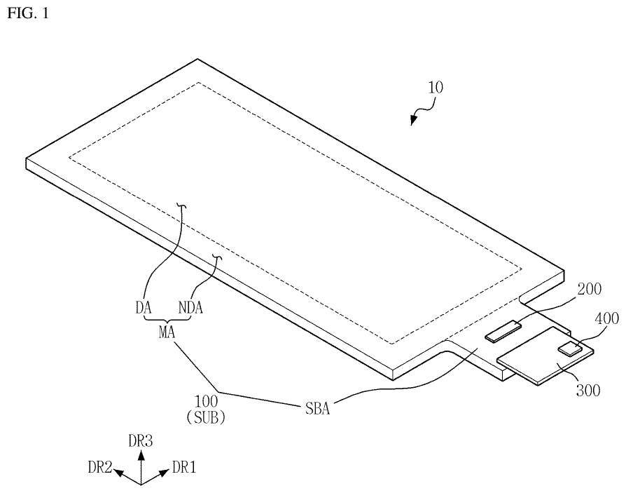

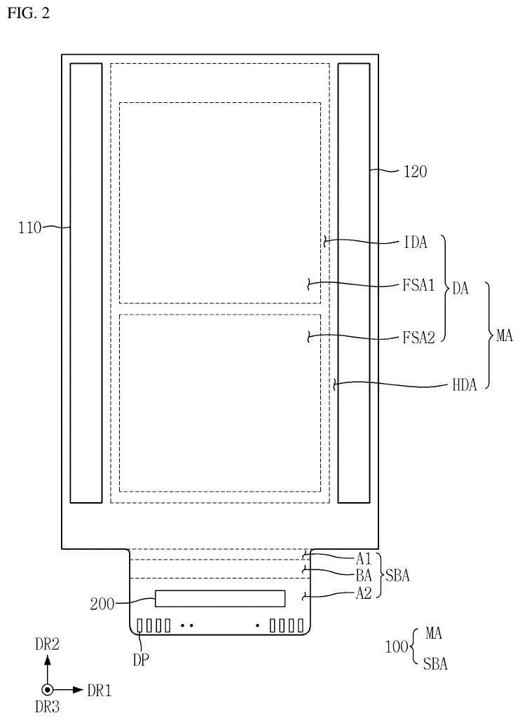

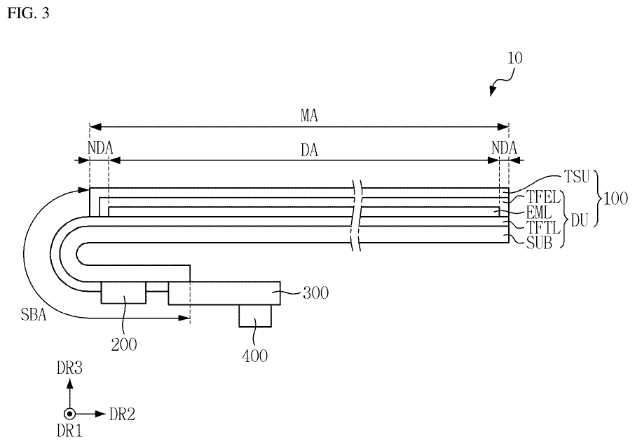

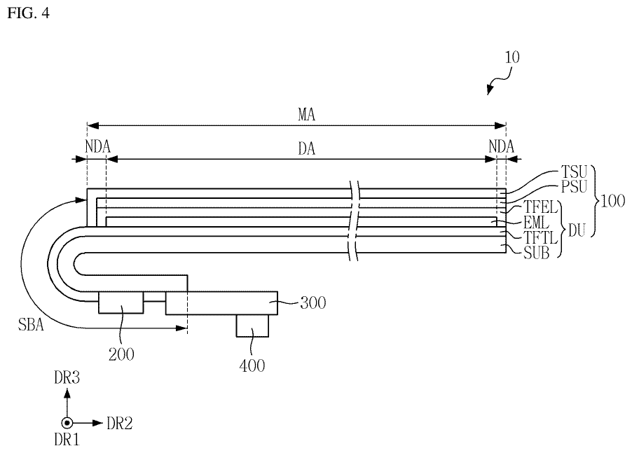

FIG. 1 is an example of a perspective view of a display device according to an embodiment of the present inventive concept. FIG. 2 is an example of a plan view illustrating an arrangement structure of a display panel and a main driving circuit as shown in FIG. 1. FIGS. 3 and 4 are examples of a side view illustrating the configuration of the display device shown in FIG. 1.

Referring to FIGS. 1 and 2, according to some embodiments, a display device 10 may be integrated into portable electronic devices such as a mobile phone, a smartphone, a tablet personal computer, a mobile communication terminal, an electronic organizer, an electronic book, a portable multimedia player (PMP), a navigation system, an ultra-mobile PC (UMPC), or the like. In some cases, the display device 10 may be integrated into a display unit of a television, a laptop, a monitor, a billboard, or an Internet-of-Things (IoT) terminal. In some cases, the display device 10 may be integrated into wearable devices such as a smart watch, a watch phone, a glasses type display, or a head mounted display (HMD). In some cases, the display device 10 may be integrated into a dashboard of a vehicle, a center fascia of a vehicle, a center information display (CID) disposed on a dashboard of a vehicle, a room mirror display in place of side mirrors of a vehicle, or a display disposed on a rear surface of a front seat for rear seat entertainment of a vehicle.

The display device 10 may be a light emitting display device such as an organic light emitting display device using an organic light emitting diode (OLED), a quantum dot light emitting display including a quantum dot light emitting layer, an inorganic light emitting display including an inorganic semiconductor, and a micro light emitting display using a micro or nano light emitting diode (LED). According to some embodiments, the display device 10 includes an organic light emitting display device, however, the present disclosure is not necessarily limited thereto.

Referring to FIGS. 1 and 3, the display device 10 includes a display panel 100, a main driving circuit 200, a touch sensing unit TSU, a circuit board 300, and a touch driving circuit 400. In some embodiments, the display device 10 further includes a pressure sensing unit PSU as shown in FIG. 4.

In the plan view, the display panel 100 may be formed in a rectangular shape having short sides measured in a first direction DR1 and long sides measured in a second direction DR2, where the second direction is perpendicular to the first direction DR1. A corner of the display panel 100 may be right-angled or rounded with a predetermined curvature. However, the planar shape of the display panel 100 is not necessarily limited to the rectangular shape. For example, the planar shape of the display panel 100 may be formed in another polygonal shape, a circular shape, or an elliptical shape. The display panel 100 may be formed to be flat, but is not necessarily limited thereto. For example, the display panel 100 may include a curved portion formed at left and right ends and having a constant curvature or a varying curvature. In some cases, the display panel 100 may be formed flexibly so that the display panel 100 can be curved, bent, folded, or rolled.

According to some embodiments, a substrate SUB of the display panel 100 may include a main region MA and a sub-region SBA. For example, the main region MA may include a display area DA for displaying an image and a non-display area NDA that is a peripheral area of the display area DA. In some cases, the non-display area NDA surrounds the display area DA.

The non-display area NDA may be disposed adjacent to the display area DA. The non-display area NDA may be an area outside the display area DA. The non-display area NDA may be disposed to surround the display area DA. The non-display area NDA may be an edge region of the display panel 100.

The display area DA includes display pixels and light sensing pixels. For example, the display pixels may be configured to display an image, and light sensing pixels may be configured to sense light reflected from a body part of a user, such as a finger. In some cases, the display area DA may further include infrared light emitting pixels that emit infrared light.

The display area DA may occupy most of the main region MA. The display area DA may be disposed at the center of the main region MA. In some cases, the display area DA may be divided into an image display area IDA in which the display pixels are disposed without the light sensing pixels, and a plurality of biometric information measurement areas FSA1 and FSA2 in which both the display pixels and the light sensing pixels are disposed. For example, the light sensing pixels formed to detect light incident or reflected from the front surface may be disposed together with the display pixels in a predetermined part of the biometric information measurement areas FSA1 and FSA2 in the display area DA of the display panel 100. An example in which the display pixels and the light sensing pixels are alternately arranged in the display area DA is described below.

Referring to FIGS. 2 and 3, the sub-region SBA may protrude from one side of the main region MA in the second direction DR2. The length of the sub-region SBA measured in the second direction DR2 may be less than the length of the main region MA measured in the second direction DR2. The length of the sub-region SBA measured in the first direction DR1 may be substantially equal to or less than the length of the main region MA measured in the first direction DR1.

The sub-region SBA may include a first region A1, a second region A2, and a bending area BA. For example, the first region A1 is a region protruding from one side of the main region MA in the second direction DR2. One side of the first region A1 may be in contact with the non-display area NDA of the main region MA, and the other side (or the opposite side) of the first region A1 may be in contact with the bending area BA. In some cases, for example, the bending area BA is disposed between the first region A1 and the second region A2.

The second region A2 is a region on which pads DP and the main driving circuit 200 are disposed. The main driving circuit 200 may be attached to, or electrically connected to, driving pads of the second region A2 using a conductive adhesive member such as an anisotropic conductive layer. The circuit board 300 may be attached to, or electrically connected to, the pads DP of the second region A2 using a conductive adhesive member. One side of the second region A2 may be in contact with the bending area BA.

The bending area BA is an area that can be bent. When the bending area BA is bent, the second region A2 may be disposed under the first region A1 and under the main region MA. The bending area BA may be disposed between the first region A1 and the second region A2. One side of the bending area BA may be in contact with the first region A1, and the other side (e.g., the opposite side) of the bending area BA may be in contact with the second region A2.

As shown in FIG. 3, the sub-region SBA may be bent. For example, a portion of the sub-region SBA may be disposed under the main region MA. The sub-region SBA may overlap the main region MA in a third direction DR3. In some cases, the main driving circuit 200, the circuit board 300, and the touch driving circuit 400 may be disposed under the main region MA of the display device 10.

The touch sensing unit TSU is configured to sense a body part such as a finger, an electronic pen, or the like. The touch sensing unit TSU is formed or disposed on the topmost layer of the display panel 100. The touch sensing unit TSU may include a plurality of touch electrodes to sense a user's touch in a capacitive manner.

The touch sensing unit TSU includes a plurality of touch electrodes arranged to intersect each other in the first and second directions DR1 and DR2. For example, the plurality of touch electrodes include a plurality of driving electrodes arranged to be spaced apart from each other in parallel in the first direction DR1, and a plurality of sensing electrodes arranged to be spaced apart from each other in parallel in the second direction DR2. In some cases, the plurality of sensing electrodes intersects the plurality of driving electrodes with an organic material layer or an inorganic material layer interposed therebetween. The plurality of driving electrodes and the plurality of sensing electrodes may be formed to extend to a wiring region between display pixels SP and light sensing pixels arranged in the display area DA. In some cases, the plurality of driving electrodes and the plurality of sensing electrodes might not overlap the display pixels and the light sensing pixels. The plurality of driving electrodes and the plurality of sensing electrodes form a mutual capacitance, and transmit touch sensing signals that vary according to a touch of a user to the touch driving circuit 400.

The touch driving circuit 400 supplies touch driving signals to the plurality of driving electrodes and receives the touch sensing signals from the plurality of sensing electrodes RE. Then, the change in the mutual capacitance between the driving electrodes and the sensing electrodes is sensed based on the change in the magnitude of the touch sensing signal. The touch driving circuit 400 generates touch data according to the change in the mutual capacitance between the driving electrodes and the sensing electrodes and obtains positions where a touch is sensed. The touch driving circuit 400 generates touch data including a plurality of touch positions and a plurality of touch position coordinates according to the change in in the mutual capacitance between the touch nodes in the touch sensing unit TSU. Accordingly, the touch driving circuit 400 may supply coordinate data of the plurality of touch positions where the touch is sensed to the main driving circuit 200.

FIG. 4 is an example of a side view of illustrating the configuration of the display device as shown in FIG. 1. For example, the display device 10 in FIG. 4 includes pressure sensing unit PSU disposed under the touch sensing unit TSU.

The pressure sensing unit PSU is configured to sense the pressure applied by a body part, such as a finger or the like, may be disposed or formed on the front surface (e.g., the top most layer in the cross-sectional view) of the display panel 100. In some cases, for example, pressure sensing unit PSU is disposed between the touch sensing unit TSU and the substrate SUB.

In some embodiments, the display device includes the pressure sensing unit PSU to detect absolute blood pressure-related measurement values. In some embodiments, the display device might not include the pressure sensing unit PSU to detect relative blood pressure-related measurement values. Therefore, as shown in FIG. 3, the pressure sensing unit PSU might not be formed.

Referring to FIG. 4, the pressure sensing unit PSU may be formed of a transparent sheet type in which a plurality of transparent electrodes are arranged in vertical and horizontal directions. In some cases, the pressure sensing unit PSU may be disposed on the front surface (or upper layer in the cross-sectional view) of the main area MA. In some cases, the pressure sensing unit PSU may be disposed or formed inside or on the front portion of the display panel 100. For example, the pressure sensing unit PSU may be formed between the touch sensing unit TSU and the substrate SUB.

The pressure sensing unit PSU includes a plurality of pressure sensing electrodes arranged to intersect each other in the first direction DR1 and the second direction DR2. The plurality of pressure sensing electrodes include a plurality of lower electrodes arranged to be spaced apart from each other in parallel in the first direction DR1, and a plurality of upper electrodes arranged to be spaced apart from each other in parallel in the second direction DR2. In some cases, the plurality of upper electrodes intersects the plurality of lower electrodes with a transparent inorganic (or organic) material layer interposed therebetween. The plurality of lower electrodes and the plurality of upper electrodes form a self-capacitance with a transparent inorganic (or organic) material layer interposed therebetween. In some cases, the plurality of lower electrodes and the plurality of upper electrodes transmit pressure sensing signals that vary according to a touch pressure to the touch driving circuit 400.

When the pressure sensing unit PSU is disposed on the front surface of the display panel 100, the pressure sensing electrodes (e.g., the plurality of lower electrodes and the plurality of upper electrodes) of the pressure sensing unit PSU may be formed to extend to the wiring region between the display pixels and the light sensing pixels arranged in the display area DA. In some cases, the pressure sensing electrodes might not overlap the display pixels and the light sensing pixels. In some cases, the touch driving circuit 400 may supply a reference voltage to the lower electrodes of the pressure sensing unit PSU, and receive pressure sensing signals from the upper electrodes thereof, thereby sensing the changes in the self-capacitance of the pressed areas using the pressure sensing signals. Accordingly, the touch driving circuit 400 may generate pressure data according to the amount of change in the self-capacitance and sensing coordinate data of a position where a touch is sensed and supply the generated data to the main driving circuit 200. The pressure sensing unit PSU may be applied to various other structures in addition to the structure using the pressure sensing electrodes, and is not limited to the description of FIGS. 3 and 4.

The circuit board 300 may be connected to one end of the sub-region SBA opposite from the end that is connected to the main area MA. Thus, the circuit board 300 may be electrically connected to the display panel 100 and the main driving circuit 200. The display panel 100 and the main driving circuit 200 may receive digital video data, timing signals, and driving voltages through the circuit board 300. The circuit board 300 may be a flexible printed circuit board, a printed circuit board, or a flexible film such as a chip on film.

The main driving circuit 200 may generate electrical signals such as data voltages and control signals for driving the display panel 100. The touch driving circuit 400 and the main driving circuit 200 may be formed as an integrated circuit (IC) and attached onto the display panel 100 or the circuit board 300 by a chip on glass (COG) method, a chip on plastic (COP) method, or an ultrasonic bonding method, but the present disclosure is not necessarily limited thereto. For example, the touch driving circuit 400 and the main driving circuit 200 may be attached onto the circuit board 300 by a chip on film (COF) method. In some cases, the touch driving circuit 400 and the main driving circuit 200 may be separately attached to the circuit board 300.

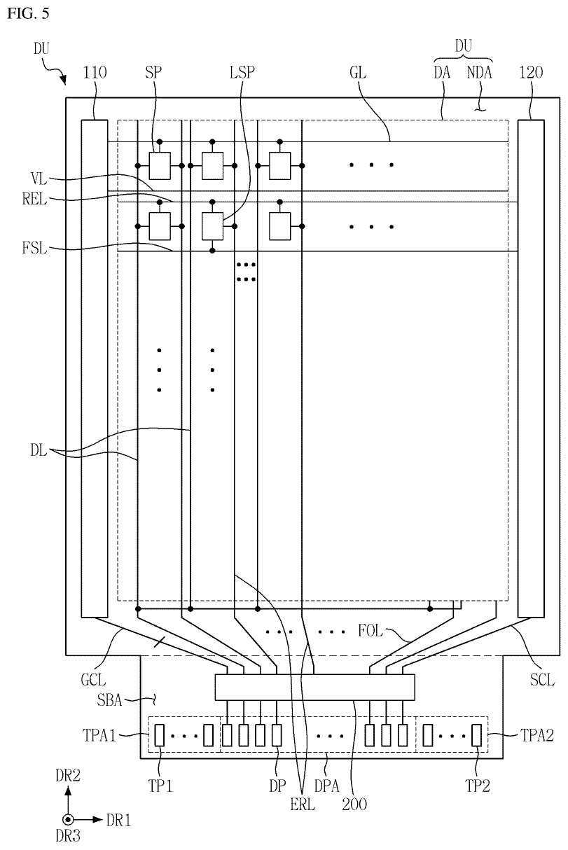

FIG. 5 is an example of a schematic layout diagram of the display panel illustrated in FIGS. 1 to 4. For example, FIG. 5 is a layout diagram illustrating the display area DA and the non-display area NDA of a display module DU before the touch sensing unit TSU is formed thereon.

Referring to FIG. 5 and FIG. 4, a display scan driver 110, a light sensing scan driver 120, and a main driving circuit 200 may be disposed on the display panel 100 of the display device 10. In some embodiments, a touch driving circuit 400 and a power supply unit may be disposed on the circuit board 300, where the circuit board 300 is connected to the display panel 100. For example, the main driving circuit 200 and the touch driving circuit 400 may be integrally formed as a one-chip type, and may be mounted onto the display panel 100 or the circuit board 300. However, hereinafter, for simplicity of functional description, an example in which the main driving circuit 200 and the touch driving circuit 400 are formed as different integrated circuits is described.

Referring to FIG. 5, the display panel 100 may include the display pixels SP, the light sensing pixels LSP, display scan lines GL, emission control lines VL, data lines DL, sensing scan lines FSL, sensing reset lines REL, and light sensing lines ERL that are disposed in the display area DA. Each of the display scan driver 110 and the light sensing scan driver 120 are disposed in the non-display area NDA.

The display scan lines GL sequentially supply the display scan signals applied in units of horizontal lines from the display scan driver 110 to the display pixels SP and light sensing pixels LSP for each horizontal line. The display scan lines GL may extend in the first direction DR1 and may be spaced apart from each other in the second direction DR2, where the second direction intersects the first direction DR1.

The emission control lines VL sequentially supply the emission control signals applied in units of horizontal lines from the display scan driver 110 to the display pixels SP and the light sensing pixels LSP for each horizontal line. The emission control lines VL may extend in the first direction DR1 and may be parallel with the display scan lines GL. In some cases, the emission control lines VL may be spaced apart from each other in the second direction DR2.

The data lines DL may supply the data voltage from the main driving circuit 200 to the plurality of display pixels SP. The plurality of data lines DL may extend in the second direction DR2 and may be spaced apart from each other in the first direction DR1.

The light sensing scan lines FSL sequentially supply the sensing scan signals applied in units of horizontal lines from the light sensing scan driver 120 to the plurality of light sensing pixels LSP. The light sensing scan line FSL may extend in the first direction DR1 and may be spaced apart from each other in the second direction DR2.

The sensing reset lines REL sequentially supply the sensing reset signals applied in units of horizontal lines from the light sensing scan driver 120 to the plurality of light sensing pixels LSP for each horizontal line. The sensing reset lines REL may extend in the first direction DR1 and may be parallel with the light sensing scan lines FSL. In some cases, the sensing reset lines REL may be spaced apart from each other in the second direction DR2.

The light sensing lines ERL are connected between the light sensing pixels LSP and the main driving circuit 200 to supply the light sensing signals outputted from the light sensing pixels LSP to the main driving circuit 200. The light sensing lines ERL may be disposed and extended in the second direction DR2 based on the arrangement direction of the main driving circuit 200, and may be spaced apart from each other in the first direction DR1.

The non-display area NDA may surround the display area DA. The non-display area NDA may include the display scan driver 110, the light sensing scan driver 120, fan-out lines FOL, gate control lines GCL, and light sensing control lines SCL.

The display pixels SP and the light sensing pixels LSP may form a first unit pixel and may be arranged in a matrix form in the first direction DR1 and the second direction DR2 in the display area DA. When at least one infrared light emitting pixel is additionally disposed in the display area DA, the display pixels SP and at least one infrared light emitting pixel may form a second unit pixel, and the second unit pixels and the first unit pixels may be alternately arranged in a matrix form in the display area DA. For example, the first unit pixel includes a display pixel SP and a light sensing pixel LSP. For example, the second unit pixel includes a display pixel SP and an infrared light emitting pixel.

For example, three display pixels SP that respectively display red, green, and blue light, and one light sensing pixel LSP may form one first unit pixel. In some embodiments, three display pixels SP that respectively display red, green, and blue light, and one infrared light emitting pixel may form one second unit pixel. In some cases, each of the pixels of the first unit pixel may be arranged in a quadrant of the first unit pixel. In some cases, each of the pixels of the second unit pixel may be arranged in a quadrant of the second unit pixel. The first unit pixels and the second unit pixels may be alternately arranged in horizontal or vertical stripes in a matrix form. In some cases, the first unit pixels and the second unit pixels may be alternately arranged in a zigzag shape in a plan view, and may be arranged in a matrix form in one diagonal direction.

Each of the red, green, and blue display pixels SP and the infrared light emitting pixels may be connected to one of the display scan lines GL and one of the emission control lines VL. During an image display period, the red, green, and blue display pixels SP may receive the data voltage of the data line DL based on the display scan signal of the display scan line GL and the emission control signal of the emission control line VL, and may supply a driving current to the light emitting element based on the data voltage, thereby emitting light. For example, during the measurement period to measure biometric information such as a blood pressure, a heart rate, oxygen saturation, blood vessel elasticity, etc., the display pixels SP displaying at least one color among the red, green, and blue display pixels SP may selectively receive the data voltage for light emission together with the display scan signal and the emission control signal and display the RGB light. In some cases, during the measurement period to measure biometric information such as a blood pressure, a heart rate, etc., the infrared light emitting pixels may selectively receive the data voltage for light emission together with the display scan signal and the emission control signal and display infrared light.

The light sensing pixels LSP may be alternately arranged with the red, green, and blue display pixels SP in a vertical or horizontal direction. Each of the light sensing pixels LSP may be connected to one of the light sensing scan lines FSL, one of the sensing reset lines REL, and one of the light sensing lines ERL. During the measurement period to measure the biometric information, each of the light sensing pixels LSP is reset in response to the sensing reset signal from the sensing reset lines REL, and may generate the light sensing signal corresponding to the light amount of the reflected lights incident from the front surface. In some cases, each of the light sensing pixels LSP may transmit the light sensing signal to the light sensing line ERL in response to the sensing scan signal from the light sensing scan lines FSL.

In some cases, each of the light sensing pixels LSP may be connected to one of the display scan lines GL in units of horizontal lines. Each of the light sensing pixels LSP may generate the light sensing signal corresponding to the light amount of the reflected lights incident from the front surface, and may output the light sensing signal to the light sensing line ERL in response to the display scan signal inputted through the display scan line GL.

A display scan driver 110 may be disposed in the non-display area NDA. Although the display scan driver 110 is illustrated to be disposed on one side (e.g., left side) of the display panel 100, it is not necessarily limited to the drawings herein. For example, the display scan driver 110 may be disposed on both sides (e.g., left and right sides) of the display panel 100.

The display scan driver 110 may be electrically connected to the main driving circuit 200 through the gate control lines GCL. The display scan driver 110 receives the scan control signal from the main driving circuit 200, and sequentially generates the display scan signals in units of horizontal line driving periods according to the scan control signal and sequentially supplies the display scan signals to the display scan lines GL. For example, the display scan driver 110 may sequentially generate the emission control signals based on the scan control signal from the main driving circuit 200 and sequentially supply the emission control signals to the emission control lines VL.

The gate control line GCL may extend from the main driving circuit 200 to the display scan driver 110 based on the arrangement position of the display scan driver 110. The gate control line GCL may supply the scan control signal received from the main driving circuit 200 to the display scan driver 110.

The light sensing scan driver 120 may be disposed on another side of the non-display area NDA different from that of the display scan driver 110. FIG. 5 illustrates that the light sensing scan driver 120 is disposed on the other side (for example, the right side) of the display panel 100, but the present disclosure is not necessarily limited thereto. The light sensing scan driver 120 may be electrically connected to the main driving circuit 200 through the light sensing control lines SCL. The light sensing scan driver 120 receives the light sensing control signal from the main driving circuit 200, and sequentially generates the reset control signals and the light sensing scan signals in units of horizontal line driving periods based on the light sensing control signal. Then, the sequentially generated reset control signals are sequentially supplied to the sensing reset lines REL. For example, the light sensing scan driver 120 may sequentially generate the sensing scan signals based on the light sensing control signal from the main driving circuit 200 and sequentially supply the sensing scan signals to the sensing scan lines FSL.

The light sensing control line SCL may extend from the main driving circuit 200 to the light sensing scan driver 120 based on the arrangement position of the light sensing scan driver 120. The light sensing control line SCL may supply the light sensing control signal received from the main driving circuit 200 to the light sensing scan driver 120.

The sub-region SBA may include the main driving circuit 200, a display pad area DPA, and first and second touch pad areas TPA1 and TPA2. The display pad area DPA, the first touch pad area TPA1, and the second touch pad area TPA2 may be disposed at the edge of the sub-region SBA. In some cases, the display pad area DPA may be disposed between the first touch pad area TPA1 and the second touch pad area TPA2 in the first direction DR1. In some cases, the main driving circuit 200 may be disposed between the non-display area NDA and the display pad area DPA in the second direction DR2. The display pad area DPA, the first touch pad area TPA1, and the second touch pad area TPA2 may be electrically connected to the circuit board 300 by using an anisotropic conductive layer or a low-resistance high-reliability material such as SAP.

The fan-out lines FOL may extend from the main driving circuit 200 to the display area DA. For example, the fan-out lines FOL are connected such that the data voltage received from the main driving circuit 200 may be supplied to each of the plurality of data lines DL. In some cases, the gate control line GCL and the light sensing control line SCL may extend from the main driving circuit 200 to the display scan driver 110 and the light sensing scan driver, respectively, in the non-display area NDA.

The main driving circuit 200 may output signals and voltages for driving the display panel 100 to the fan-out lines FOL. The main driving circuit 200 may supply a data voltage to the data line DL through the fan-out lines FOL. The data voltage may be supplied to the plurality of display pixels SP to determine the luminance of the display pixels SP. The main driving circuit 200 may supply the scan control signal to the display scan driver 110 through the gate control line GCL.

The main driving circuit 200 receives the light sensing signals from the light sensing pixels LSP through the light sensing lines ERL, and detects a photoplethysmography signal among biometric signals based on the change in the magnitudes of the light sensing signals. For example, the light sensing signals includes pulse wave signals.

In addition to the pulse wave signal, the biometric signals may further include an EMG signal, an EEG signal, etc. However, an example in which the main driving circuit 200 detects and analyzes pulse wave signals among biometric signals to measure the biometric information is described below. The biometric information of the user includes information such as blood pressure, heart rate, heart rate variability, respiratory rate, blood vessel elasticity, occurrence or non-occurrence of a cardiovascular disease, oxygen saturation, etc.

The main driving circuit 200 receives biometric information such as pulse wave signals of the user through a separate biometric information detection device such as a cuff-type blood pressure measuring device which measures biometric signals like pulse wave signals by applying pressure to the body part of a user.

The main driving circuit 200 receives pulse wave signals and biometric information detected through a left arm, and guides a pulse wave signal detection process with an application program screen so that the pulse wave signals may be detected through a right finger. The main driving circuit 200 detects the pulse wave signals in real time through the right finger or other body parts. Accordingly, the main driving circuit 200 numerically stores the characteristics of a left arm pulse wave signals, that are amplitude and pulse width range (numerical range) of the left arm pulse wave signals, the systolic and diastolic cycles thereof, a systolic blood pressure change width range (numerical range) thereof, a systolic cycle thereof, and a diastolic cycle change range (numerical range) thereof detected through a separate biometric information detection device. In some cases, the characteristics of a pulse wave signals, that are amplitude and pulse width range (numerical range) of the right finger pulse wave signals, the systolic and diastolic cycles thereof, a systolic blood pressure change width range (numerical range) thereof, a systolic cycle thereof, and a diastolic cycle change range (numerical range) thereof detected through the right finger, are numerically stored. The main driving circuit 200 substitutes the numerical characteristic information of the right finger pulse wave signals based on the numerical characteristic information of the left arm pulse wave signals detected through the biometric information detection device, and sets reference correction values (hereinafter, first reference correction values) with respect to the right finger based on the average or difference value. In the process of setting the first reference correction values, the left arm and right finger pulse wave signals detection process may be repeated multiple times (e.g., at least three times) to extract the reference correction values, and the first reference correction values may be set as the average value of the reference values that are extracted. In some cases, the repetitive extraction process is to ensure the accuracy of the first reference correction value. However, embodiments are not necessarily limited thereto. For example, the second reference correction value may be determined based on a single extraction process.

Similarly, the main driving circuit 200 receives pulse wave signals and biometric information detected through the right hand of a user and guides a pulse wave signal detection process with the application program screen so that the pulse wave signals may be detected through the left finger of the user. The main driving circuit 200 detects the pulse wave signals in real time through the left finger. Accordingly, the main driving circuit 200 numerically stores the characteristics of a right arm pulse wave signals, that are amplitude and pulse width range (numerical range) of the right arm pulse wave signals, the systolic and diastolic cycles thereof, a systolic blood pressure change width range (numerical range) thereof, a systolic cycle thereof, and a diastolic cycle change range (numerical range) thereof detected through a separate biometric information detection device, etc. In some cases, the characteristics of a pulse wave signals, that are amplitude and pulse width range (numerical range) of the left finger pulse wave signals, the systolic and diastolic cycles thereof, a systolic blood pressure change width range (numerical range) thereof, a systolic cycle thereof, and a diastolic cycle change range (numerical range) thereof detected through the left finger are numerically stored. The main driving circuit 200 substitutes the numerical characteristic information of the left finger pulse wave signals based on the numerical characteristic information of the right arm pulse wave signals detected through the biometric information detection device, and sets a reference correction values (hereinafter, second reference correction values) with respect to the left finger based on the average or difference value. In the process of setting the second reference correction values, the right arm and left finger pulse wave signals detection process may be repeated multiple times (e.g., at least three times) to extract the reference correction values, and the second reference correction values may be set as the average value of the reference values that are extracted. In some cases, the repetitive extraction process is to ensure the accuracy of the second reference correction value. However, embodiments are not necessarily limited thereto. For example, the second reference correction value may be determined based on a single extraction process.

The main driving circuit 200 stores the first and second reference correction values, respectively, and guides the pulse wave signal detection process with the application program screen so that the pulse wave signals may be detected through the both fingers of the left and right hands when measurement of the biometric information of the user through the application program is requested. Then, each of the pulse wave signals are detected through both fingers.

The main driving circuit 200 analyzes pulse wave signals detected in real time through the right fingers based on the preset and pre-stored first reference correction values using a preset machine learning algorithm, thereby measuring the first biometric information of blood pressure, heart rate, heart rate variability, etc. In some cases, the main driving circuit 200 analyzes pulse wave signals detected in real time through the left fingers based on the preset and pre-stored second reference correction values using a preset machine learning algorithm, thereby measuring the second biometric information of blood pressure, heart rate, heart rate variability, etc.

For example, the main driving circuit 200 may select the biometric information in which the blood pressure level is measured to be higher among the first and second biometric information as the user's real-time biometric information and display the selected biometric information on the application program screen. In some cases, the main driving circuit 200 may generate digital video data based on the touch coordinates of a touch coordinate data from the touch driving circuit 400, or execute an application indicated by an icon displayed on the user's touch coordinates.

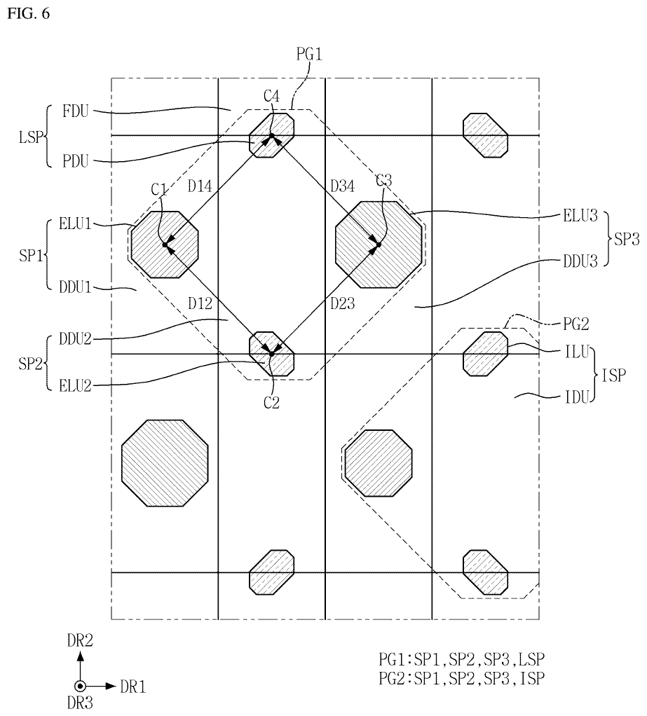

FIG. 6 is an example of a layout diagram illustrating a display area according to an embodiment. Referring to FIG. 6, the display area DA may include the display pixels SP, infrared light emitting pixels ISP, and the light sensing pixels LSP. For example, the display pixels SP may include the first display pixels SP1, the second display pixels SP2, and the third display pixels SP3.

The light sensing pixel LSP, the first display pixel SP1, the second display pixel SP2, and the third display pixel SP3 may be formed as a first unit pixel PG1. For example, the infrared light emitting pixel ISP, the first display pixel SP1, the second display pixel SP2, and the third display pixel SP3 may be formed as a second unit pixel PG2.

The first and second unit pixels PG1 and PG2 may be minimum unit display pixels capable of displaying light, and each first unit pixel PG1 may sense light. The first unit pixels PG1 and the second unit pixels PG2 may be alternately arranged in a zigzag shape in a plan view, and may be arranged in a matrix form in a diagonal direction. For example, the first unit pixels PG1 and the second unit pixels PG2 may be alternately arranged in horizontal or vertical stripes in a matrix form in a plan view.

The first display pixel SP1 may include a first light emitting portion ELU1 that emits first light, and a first pixel driver DDU1 for applying a driving current to the light emitting element of the first light emitting portion ELU1. The first light may be light of a red wavelength band. For example, the main peak wavelength of the first light may be located at approximately 600 nm to 750 nm.

The second display pixel SP2 may include a second light emitting portion ELU2 that emits second light, and a second pixel driver DDU2 for applying a driving current to the light emitting element of the second light emitting portion ELU2. The second light may be light of a blue wavelength band. For example, the main peak wavelength of the second light may be located at approximately 370 nm to 460 nm.

The third display pixel SP3 may include a third light emitting portion ELU3 that emits third light, and a third pixel driver DDU3 for applying a driving current to the light emitting element of the third light emitting portion ELU3. For example, the third light may be light of a green wavelength band. For example, the main peak wavelength of the third light may be located at approximately 480 nm to 560 nm.

The infrared light emitting pixel ISP may include an infrared light emitting portion ILU emitting light of an infrared wavelength band and an infrared light pixel driver IDU for applying a driving current to the light emitting element of the infrared light emitting portion ILU. The main peak wavelength of the infrared light may be located at approximately 750 nm to 1 mm. In some cases, the light sensing pixel LSP includes the light sensing portion PDU and the sensing driver FDU.

In the first unit pixel GP1, the first to third pixel drivers DDU1 to DDU3 may be arranged in a preset order in the first direction DR1. In some cases, any one of the first to third pixel drivers DDU1 to DDU3 may be disposed in the first direction DR1 of another adjacent pixel driver. For example, the sensing driver FDU may be disposed in the first direction DR1 of any one of the first to third pixel drivers DDU1 to DDU3. In some cases, the sensing driver FDU may be disposed in the second direction DR2 of any one of the first to third pixel drivers DDU1 to DDU3.

The first pixel drivers DDU1 adjacent to each other in the data line direction may be disposed in the second direction DR2. The second pixel drivers DDU2 adjacent to each other in the direction of the data line DL may be disposed in the second direction DR2. Similarly, the sensing drivers FDU adjacent to each other in the direction of the data line DL may also be disposed in the second direction DR2.

The first light emitting portion ELU1, the second light emitting portion ELU2, the third light emitting portion ELU3, the infrared light emitting portion ILU, and the light sensing portion PDU may have a rectangular, octagonal, or rhombic planar shape, but the present disclosure is not necessarily limited thereto. The first light emitting portion ELU1, the second light emitting portion ELU2, the third light emitting portion ELU3, the infrared light emitting portion ILU, and the light sensing portion PDU may have another polygonal planar shape other than a rectangle, an octagon, and a rhombus.

In some cases, due to the arrangement position and planar shape of the first light emitting portion ELU1, the second light emitting portion ELU2, the third light emitting portion ELU3, and the light sensing portion PDU, a distance D12, a distance D23, a distance D14, and a distance D34 may be substantially the same. For example, a distance D12 is measured between a center C1 of the first light emitting portion ELU1 and a center C2 of the second light emitting portion ELU2 adjacent to each other. For example, a distance D23 is measured between the center C2 of the second light emitting portion ELU2 and a center C3 of the third light emitting portion ELU3 adjacent to each other. For example, a distance D14 is measured between the center C1 of the first light emitting unit ELU1 and a center C4 of the light sensing portion PDU adjacent to each other in another direction. For example, a distance D34 is measured between a center C4 of the light sensing portion PDU and the center C3 of the third light emitting portion ELU3.

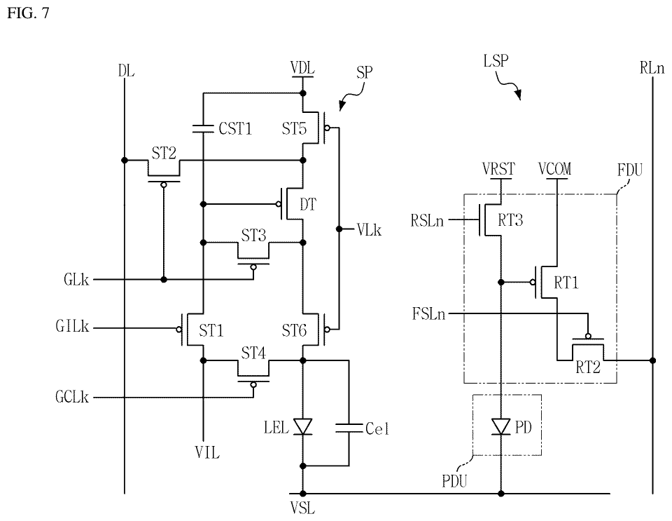

FIG. 7 is an example of a circuit diagram illustrating a display pixel and a light sensing pixel according to an embodiment of the present inventive concept. Referring to FIG. 7, each display pixel SP may be connected to a kth display initialization line GILk, a kth display scan line GLk, a kth display control line GCLk, and a kth emission control line VLk. In some cases, the display pixel SP may be connected to a first driving voltage line VDL to which the first driving voltage is supplied, a second driving voltage line VSL to which the second driving voltage is supplied, and a third driving voltage line VIL to which the third driving voltage is supplied. Hereinafter, the alphabet letters k, n, etc. used in place of numbers are defined to be positive integers excluding 0.

The display pixel SP may include a light emitting portion ELU and a pixel driver DDU. The light emitting portion ELU may include a light emitting element LEL. The pixel driver DDU may include a driving transistor DT, switch elements, and a capacitor CST1. The switch elements include the first to sixth transistors ST1, ST2, ST3, ST4, ST5, and ST6.

The driving transistor DT may include a gate electrode, a first electrode, and a second electrode. The driving transistor DT controls a drain-source current Ids (hereinafter, referred to as “driving current”) flowing between the first electrode and the second electrode based on a data voltage applied to the gate electrode. The driving current Ids flowing through a channel of the driving transistor DT is proportional to the square of the difference between a threshold voltage and a voltage Vsg between the first electrode and the gate electrode of the driving transistor DT, as shown in Eq. (1).

$$Ids = {k}^{′} \times {( Vsg - Vth )}^{2} \qquad ( 1 )$$

In Eq. (1), k′ is a proportional coefficient determined by the structure and physical characteristics of the driving transistor, Vsg is a voltage measured between the first electrode and the gate electrode of the driving transistor, and Vth is a threshold voltage of the driving transistor.

The light emitting element LEL emits light by the driving current Ids. As the driving current Ids increases, the amount of light emitted from the light emitting element LEL may increase.

The light emitting element LEL may be an organic light emitting diode including an organic light emitting layer disposed between an anode electrode and a cathode electrode. In some cases, the light emitting element LEL may be an inorganic light emitting element including an inorganic semiconductor disposed between an anode electrode and a cathode electrode. In some cases, the light emitting element LEL may be a quantum dot light emitting element including a quantum dot light emitting layer disposed between an anode electrode and a cathode electrode. In some cases, the light emitting element LEL may be a micro light emitting element including a micro light emitting diode disposed between an anode electrode and a cathode electrode.

The anode electrode of the light emitting element LEL may be connected to a first electrode of the fourth transistor ST4 and a second electrode of the sixth transistor ST6, and the cathode electrode of the light emitting element LEL may be connected to the second driving voltage line VSL. A parasitic capacitance Cel may be formed between the anode electrode and the cathode electrode of the light emitting element LEL.

The first transistor ST1 is turned on by the display initialization signal of the kth display initialization line GILk to connect the gate electrode of the driving transistor DT to the third driving voltage line VIL. Accordingly, the third driving voltage VINT of the third driving voltage line VIL may be applied to the gate electrode of the driving transistor DT. The gate electrode of the first transistor ST1 may be connected to the kth display initialization line GILk, the first electrode of the first transistor ST1 may be connected to the gate electrode of the driving transistor DT, and the second electrode of the first transistor ST1 may be connected to the third driving voltage line VIL.

The second transistor ST2 is turned on by the display scan signal of the kth display scan line GLk to connect the first electrode of the driving transistor DT to the data line DL. Accordingly, the data voltage of the data line DL may be applied to the first electrode of the driving transistor DT. The gate electrode of the second transistor ST2 may be connected to the kth display scan line GLk, the first electrode of the second transistor ST2 may be connected to the first electrode of the driving transistor DT, and the second electrode of the second transistor ST2 may be connected to the data line DL.

The third transistor ST3 is turned on by the display scan signal of the kth display scan line GLk to connect the gate electrode of the driving transistor DT to the second electrode of the third transistor ST3. When the gate electrode of the driving transistor DT is connected to the second electrode of the third transistor ST3, the driving transistor DT is driven as a diode. The gate electrode of the third transistor ST3 may be connected to the kth display scan line GLk, the first electrode of the third transistor ST3 may be connected to the second electrode of the driving transistor DT, and the second electrode of the third transistor ST3 may be connected to the gate electrode of the driving transistor DT.

The fourth transistor ST4 is turned on by the display control signal of the kth display control line GCLk to connect the anode electrode of the light emitting element LEL to the third driving voltage line VIL. The third driving voltage of the third driving voltage line VIL may be applied to the anode electrode of the light emitting element LEL. The gate electrode of the fourth transistor ST4 is connected to the kth display control line GCLk, the first electrode of the fourth transistor ST4 is connected to the anode electrode of the light emitting element LEL, and the second electrode of the fourth transistor ST4 is connected to the third driving voltage line VIL.

The fifth transistor ST5 is turned on by the emission signal of a kth emission control line VLk to connect the first electrode of the driving transistor DT to the first driving voltage line VDL. The gate electrode of the fifth transistor ST5 is connected to the kth emission control line VLk, the first electrode of the fifth transistor ST5 is connected to the first driving voltage line VDL, and the second electrode of the fifth transistor ST5 is connected to the first electrode of the driving transistor DT.

The sixth transistor ST6 is disposed between the second electrode of the driving transistor DT and the anode electrode of the light emitting element LEL. The sixth transistor ST6 is turned on by the emission control signal of the kth emission control line VLk to connect the second electrode of the driving transistor DT to the anode electrode of the light emitting element LEL. The gate electrode of the sixth transistor ST6 is connected to the kth emission control line VLK, the first electrode of the sixth transistor ST6 is connected to the second electrode of the driving transistor DT, and the second electrode of the sixth transistor ST6 is connected to the anode electrode of the light emitting element LEL.

When both the fifth transistor ST5 and the sixth transistor ST6 are turned on, the driving current Ids of the driving transistor DT according to the data voltage applied to the gate electrode of the driving transistor DT may flow to the light emitting element LEL.

The capacitor CST1 is formed between the gate electrode of the driving transistor DT and the first driving voltage line VDL. The first capacitor electrode of the capacitor CST1 may be connected to the gate electrode of the driving transistor DT, and the second capacitor electrode of the capacitor CST1 may be connected to the first driving voltage line VDL.

When the first electrode of each of the driving transistor DT and the first to sixth transistors ST1 to ST6 is a source electrode, the second electrode of the driving transistor DT may be a drain electrode. In some cases, when the first electrode of each of the driving transistor DT and the first to sixth transistors ST1 to ST6 is a drain electrode, the second electrode of the driving transistor DT may be a source electrode.

An active layer of each of the driving transistor DT and the first to sixth transistors ST1 to ST6 may include at least one of polysilicon, amorphous silicon, and an oxide semiconductor. In FIG. 7, the first to sixth transistors ST1 to ST6, and the driving transistor DT have been described to include a P-type MOSFET, but the present disclosure is not necessarily limited thereto. For example, the first to sixth transistors ST1 to ST6, and the driving transistor DT may include an N-type MOSFET. In some cases, at least one of the first to sixth transistors ST1 to ST6 may include an N-type MOSFET.

The light sensing pixels LSP are respectively electrically connected to an n^{th} sensing reset line RSLn, an n^{th} light sensing scan line FSLn, and an n^{th} light sensing line RLn. Each of the light sensing pixels LSP may be reset by a reset signal from the n^{th} sensing reset line RSLn, and may transmit a light sensing signal to each n^{th} light sensing line RLn in response to the sensing scan signal from the n^{th} light sensing scan line FSLn.

The light sensing pixels LSP may include the light sensing portion PDU and the sensing driver FDU. For example, the light sensing portion PDU includes a light sensing element PD. For example, the sensing driver FDU includes first to third sensing transistors RT1 to RT3 and a sensing capacitor. For example, the sensing capacitor may be formed in parallel with the light sensing element PD.

The first sensing transistor RT1 of the sensing driver FDU may enable a light sensing current to flow based on the voltages of the light sensing element PD and the sensing capacitor. The amount of the light sensing current may vary based on a voltage applied to the light sensing element PD and the sensing capacitor. The gate electrode of the first sensing transistor RT1 may be connected to the second electrode of the light sensing element PD. The first electrode of the first sensing transistor RT1 may be connected to a common voltage source Vcom to which a common voltage is applied. The second electrode of the first sensing transistor RT1 may be connected to the first electrode of the second sensing transistor RT2.

When the sensing scan signal of a gate-on voltage is applied to the n^{th} light sensing scan line FSLn, the second sensing transistor RT2 may enable the sensing current of the first sensing transistor RT1 to flow to the n^{th} light sensing line RLn. For example, the n^{th} light sensing line RLn may be charged with a sensing voltage by the sensing current. The gate electrode of the second sensing transistor RT2 may be connected to the n^{th} light sensing scan line FSLn, the first electrode of the second sensing transistor RT2 may be connected to the second electrode of the first sensing transistor RT1, and the second electrode of the second sensing transistor RT2 may be connected to the n^{th} light sensing line RLn.

When a reset signal of the gate-on voltage is applied to the n^{th} sensing reset line RSLn, the third sensing transistor RT3 may reset the voltages of the light sensing element PD and the sensing capacitor to a reset voltage of a reset voltage source VRST. The gate electrode of the third sensing transistor RT3 may be connected to the sensing reset line RSL, the first electrode of the third sensing transistor RT3 may be connected to the reset voltage source VRST, and the second electrode of the third sensing transistor RT3 may be connected to the second electrode of the light sensing element PD. In some cases, the second electrode of the third sensing transistor RT3 may be connected to the gate electrode of the first sensing transistor RT1.

According to some embodiments, the first sensing transistor RT1 and the second sensing transistor RT2 each includes a P-type metal oxide semiconductor field effect transistor (MOSFET), and the third sensing transistor RT3 includes an N-type MOSFET. However, an embodiment of the present disclosure is not necessarily limited thereto. For example, the first sensing transistor RT1, the second sensing transistor RT2, and the third sensing transistor RT3 may be selectively include the same type or different types. For example, one of the first electrode and the second electrode of each of the first sensing transistor RT1, the second sensing transistor RT2, and the third sensing transistor RT3 may be the source electrode and the remaining electrode may be the drain electrode.

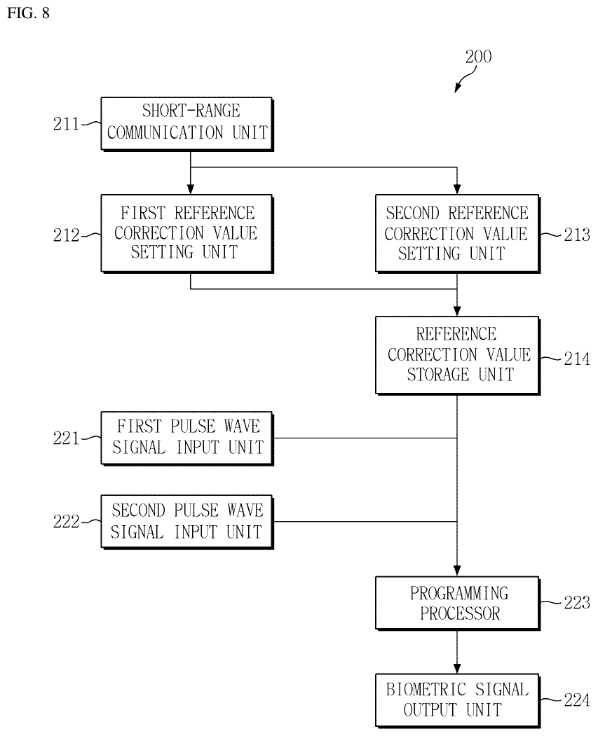

FIG. 8 is an example of a block diagram illustrating a biometric signal detection unit of a main driving circuit illustrated in FIGS. 1 to 3. Referring to FIG. 8, the main driving circuit 200 includes a short-range communication unit 211, a first reference correction value setting unit 212, a second reference correction value setting unit 213, a reference correction value storage unit 214, a first pulse wave signal input unit 221, a second pulse wave signal input unit 222, a programming processor 223, and a biometric signal output unit 224. In some embodiments, each of the units of the main driving circuit 200 may be implemented as software stored in a memory unit and executable by a processor unit, as firmware, as one or more hardware circuits, or as a combination thereof.

The short-range communication unit 211 receives the first reference pulse wave signals and the first reference biometric information from a user. For example, the first reference pulse wave signals is detected from a right finger through the display device 10. For example, the first reference biometric information is detected from the left arm through a separate biometric information detection device such as a cuff-type blood pressure measuring device. In some cases, the short-range communication unit 211 may receive the first reference pulse wave signals and the first reference biometric information transmitted through a wireless communication module such as Bluetooth and Wi-Fi. In some cases, for example, the short-range communication unit 211 receives the second reference pulse wave signals detected from a left finger and the second reference biometric information detected from the right arm.

A main processing unit of the main driving circuit 200 or the biometric signal output unit 224 of the main driving circuit 200 may guide the pulse wave signal detection process through the application program screen during the first and second reference pulse wave signal receiving periods. Accordingly, the user brings the right and left hands into contact with the display screen in which the application program is displayed and measure the first and second pulse wave signals.

The first pulse wave signal input unit 221 may first receive first pulse wave signals detected through the right finger. The first pulse wave signal input unit 221 receives light sensing signals through light sensing lines ERL during the period in which the first reference pulse wave signals and the first reference biometric information are received through the short-range communication unit 211 and transmits the first pulse wave signal based on the light sensing signals to the first reference correction value setting unit 212.

Thereafter, the second pulse wave signal input unit 222 may receive the second pulse wave signals detected through the left finger. The second pulse wave signal input unit 222 receives light sensing signals through light sensing lines ERL during the period in which the second reference pulse wave signals and the second reference biometric information are received through the short-range communication unit 211 and transmits the second pulse wave signal based on the light sensing signals to the second reference correction value setting unit 213.

The first reference correction value setting unit 212 substitutes the numerical characteristic information of the first pulse wave signals based on the numerical characteristics information of the first reference pulse wave signals and sets the first reference correction values with respect to the right finger based on the average or difference value.

For example, the first reference correction value setting unit 212 numerically stores amplitude and pulse width range (numerical range) of the first reference pulse wave signals, the systolic and diastolic cycles thereof, a systolic blood pressure change width range (numerical range) thereof, a systolic cycle thereof, and a diastolic cycle change range (numerical range) thereof. In some cases, amplitude and pulse width range (numerical range) of the first pulse wave signals, the systolic and diastolic cycles thereof, a systolic blood pressure change width range (numerical range) thereof, a systolic cycle thereof, and a diastolic cycle change range (numerical range) thereof received through the first pulse wave signal input unit 221 are detected.

The first reference correction value setting unit 212 substitutes the numerical characteristic information of the first pulse wave signals based on the numerical characteristic information of the first reference pulse wave signals, and calculates at least one value among the median value, the average value, and the difference value between the numerical characteristic information of the first pulse wave signals and the numerical characteristic information of the first reference pulse wave signals to generate the first reference correction values with respect to each numerical characteristic information. For example, as the first reference correction values are substituted with the numerical characteristic information of the first pulse wave signals based on the numerical characteristic information of the first reference pulse wave signals for at least three times, the median average, the difference average, and the total average values may be set.

In some cases, the second reference correction value setting unit 213 substitutes the numerical characteristic information of the second pulse wave signals based on the numerical characteristic information of the second reference pulse wave signals. In some cases, the second reference correction value setting unit 213 sets the second reference value with respect to the left finger based on the average or difference value.

For example, the second reference correction value setting unit 213 numerically stores amplitude and pulse width range (numerical range) of the second reference pulse wave signals, the systolic and diastolic cycles thereof, a systolic blood pressure change width range (numerical range) thereof, a systolic cycle thereof, and a diastolic cycle change range (numerical range) thereof. In some cases, amplitude and pulse width range (numerical range) of the second pulse wave signals, the systolic and diastolic cycles thereof, a systolic blood pressure change width range (numerical range) thereof, a systolic cycle thereof, and a diastolic cycle change range (numerical range) thereof received through the second pulse wave signal input unit 222 are detected.

The second reference correction value setting unit 213 substitutes the numerical characteristic information of the second pulse wave signals based on the numerical characteristic information of the second reference pulse wave signals, and calculates at least one value among the median value, the average value, and the difference value between the numerical characteristic information of the second pulse wave signals and the numerical characteristic information of the second reference pulse wave signals to generate the second reference correction values with respect to each numerical characteristics information. For example, as the second reference correction values are substituted with the numerical characteristic information of the second pulse wave signals based on the numerical characteristic information of the second reference pulse wave signals for at least three times, the median average, the difference average, and the total average values may be set.

The reference correction value storage unit 214 stores, in real time, the updated first reference correction values from the first reference correction value setting unit 212. The reference correction value storage unit 214 stores, in real time, the updated second reference correction values from the second reference correction value setting unit 213.

In some cases, the main driving circuit 200 stores each of the first and second reference correction values in the reference correction value storage unit 214. In some cases, the main driving circuit 200 guides the pulse wave signal detection process with the application program screen so that the pulse wave signals may be detected through both fingers of the left and right hands when measurement of the biometric information of the user through the application program is requested.

The programming processor 223 receives the first and second pulse wave signals in real time through the first pulse wave signal input unit 221 and the second pulse wave signal input unit 222 during the measurement period of biometric information through the application program screen.

The programming processor 223 analyzes the first and second pulse wave signals input in real time respectively based on pre-stored first and second reference correction values using a preset machine learning algorithm. For example, the programming processor 223 analyzes the first pulse wave signals detected in real time based on pre-stored first reference correction values through a preset machine learning algorithm, and generates the first biometric information such as blood pressure, heart rate, and heart rate variability.

In some cases, the programming processor 223 analyzes the second pulse wave signals detected in real time based on pre-stored second reference correction values using a preset machine learning algorithm, and generates the second biometric information such as blood pressure, heart rate, and heart rate variability.

In some cases, for example, the biometric signal output unit 224 selects one of biometric information with an abnormal level (e.g., high blood pressure level) among the first and second biometric information measured by the programming processor 223 as the real-time biometric information and displays the abnormal biometric information on the application program screen. In one aspect, the biometric signal output unit 224 may generate digital video data corresponding to touch coordinates of the user based on the touch coordinate data, or may execute an application indicated by an icon displayed on the touch coordinates of the user.

In some cases, a machine learning algorithm is a computational algorithm designed to recognize patterns, make predictions, or perform a specific task without being explicitly programmed. According to some aspects, the machine learning model is implemented as software stored in a memory unit and executable by a processor unit, as firmware, as one or more hardware circuits, or as a combination thereof. In some cases, the machine learning algorithm is performed using a machine learning model.

According to some embodiments of the present disclosure, the machine learning model includes an ANN, which is a hardware or a software component that includes a number of connected nodes (e.g., artificial neurons), which loosely correspond to the neurons in a human brain. Each connection, or edge, transmits a signal from one node to another (like the physical synapses in a brain). When a node receives a signal, the node processes the signal and then transmits the processed signal to other connected nodes. In some cases, the signals between nodes comprise real numbers, and the output of each node is computed by a function of the sum of its inputs. In some examples, nodes may determine the output using other mathematical algorithms (e.g., selecting the max from the inputs as the output) or any other suitable algorithm for activating the node. Each node and edge is associated with one or more node weights that determine how the signal is processed and transmitted.

During the training process, the one or more node weights are adjusted to increase the accuracy of the result (e.g., by minimizing a loss function that corresponds in some way to the difference between the current result and the target result). The weight of an edge increases or decreases the strength of the signal transmitted between nodes. In some cases, nodes have a threshold below which a signal is not transmitted at all. In some examples, the nodes are aggregated into layers. Different layers perform different transformations on the corresponding inputs. The initial layer is known as the input layer and the last layer is known as the output layer. In some cases, signals traverse certain layers multiple times.

In one aspect, machine learning model includes machine learning parameters. Machine learning parameters, also known as model parameters or weights, are variables that provide behaviors and characteristics of the machine learning model. Machine learning parameters can be learned or estimated from training data and are used to make predictions or perform tasks based on learned patterns and relationships in the data.

Machine learning parameters are adjusted during a training process to minimize a loss function or maximize a performance metric. The goal of the training process is to find optimal values for the parameters that allow the machine learning model to make accurate predictions or perform well on the given task.

For example, during the training process, an algorithm adjusts machine learning parameters to minimize an error or loss between predicted outputs and actual targets according to optimization techniques like gradient descent, stochastic gradient descent, or other optimization algorithms. Once the machine learning parameters are learned from the training data, the machine learning parameters are used to make predictions on new, unseen data.

According to some embodiments, the machine learning model includes a computer-implemented recurrent neural network (RNN). An RNN is a class of ANN in which connections between nodes form a directed graph along an ordered (e.g., a temporal) sequence. This enables an RNN to model temporally dynamic behavior such as predicting what element should come next in a sequence. Thus, an RNN is suitable for tasks that involve ordered sequences such as text recognition (where words are ordered in a sentence). In some cases, an RNN includes one or more finite impulse recurrent networks (characterized by nodes forming a directed acyclic graph), one or more infinite impulse recurrent networks (characterized by nodes forming a directed cyclic graph), or a combination thereof.

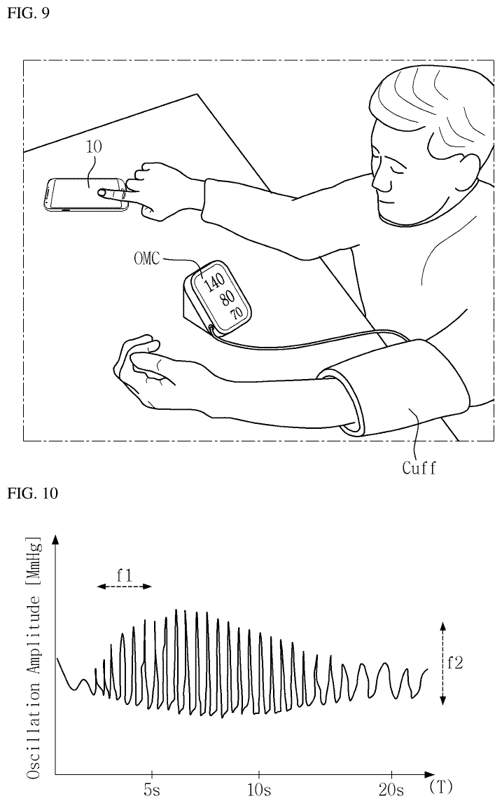

FIG. 9 is an example of a diagram illustrating a process for setting reference correction values based on a pulse wave signal detected from a right finger of a user. For example, the diagram includes display device 10 and a separate biometric information detection device OMC including a cuff. FIG. 10 is an example of a waveform diagram illustrating a detection waveform of a pulse wave signal from a biometric signal measuring device of FIG. 9. For example, the y-axis of the waveform diagram represents oscillation amplitude and the x-axis of the waveform diagram represents time.

Referring to FIGS. 9 and 10, the user uses a separate biometric information detection device OMC such as a cuff-type blood pressure measuring device and detect first reference pulse wave signals of the left arm during the period for generating the first reference correction values.

The biometric information detection device detects the first reference pulse wave signals after applying pressure to the left arm of a user, and analyzes the characteristics such as a pulse width f1 and amplitude f2 of the first reference pulse wave signals using the preset program to generate the first reference biometric information such as blood pressure, electrocardiogram, heart rate, etc.

The biometric information detection device transmits the first reference pulse wave signals and the first reference biometric information to a wireless communication module such as Bluetooth and Wi-Fi, and the main driving circuit 200 receives the first reference pulse wave signals and the first reference biometric information through the wireless communication module and the short-range communication unit 211. In some cases, the biometric information detection device directly transmits the first reference pulse wave signals and the first reference biometric information to the short-range communication unit 211.

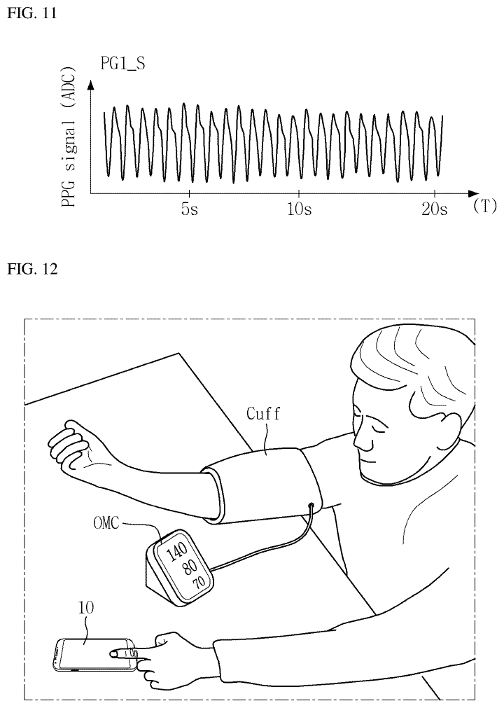

FIG. 11 is an example of a waveform diagram illustrating a detection waveform of a pulse wave signal detected through a display device of FIG. 9. For example, the y-axis of the waveform diagram represents PPG signal and the x-axis of the waveform diagram represents time.

Referring to FIG. 11, the main processing unit of the main driving circuit 200 or the biometric signal output unit 224 of the main driving circuit 200 guides the pulse wave signal detection process of the right hand through the application program screen. Accordingly, the user may bring the right hand into contact with the display screen displayed with the application program screen, where the display device 10 is able to measure first pulse wave signals PG1_S. The first pulse wave signal input unit 221 receives light sensing signals through the light sensing lines ERL of the display panel 100 and transmits the first pulse wave signals PG1_S based on the light sensing signals to the first reference correction value setting unit 212.

The first reference correction value setting unit 212 substitutes the numerical characteristic information of the first pulse wave signals PG1_S based on the numerical characteristic information of the first reference pulse wave signals, and generates first reference correction values based on at least one calculation result among the median value, the average value, and the difference value between the numerical characteristic information of the first pulse wave signals PG1_S and the numerical characteristic information of the first reference pulse wave signals.

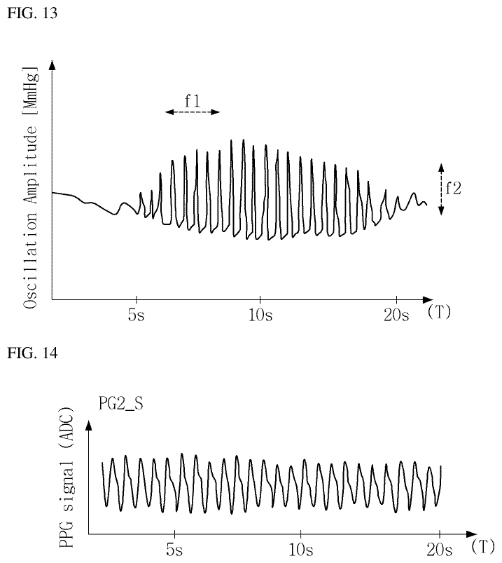

FIG. 12 is an example of a diagram illustrating a process for setting reference correction values based on a pulse wave signal detected through a left finger of a user. For example, the diagram includes display device 10 and a separate biometric information detection device OMC including a cuff. In addition, FIG. 13 is an example of a waveform diagram illustrating a detection waveform of a pulse wave signal detected through a biometric signal measuring device of FIG. 12. For example, the y-axis of the waveform diagram represents oscillation amplitude and the x-axis of the waveform diagram represents time.

Referring to FIGS. 12 and 13, the user uses a separate biometric information detection device OMC such as a cuff-type blood pressure measuring device and detects the second reference pulse wave signals of the right arm during the period of generating the second reference correction values.

The biometric information detection device detects the second reference pulse wave signals after applying pressure to the right arm of the user, and analyzes the characteristics such as a pulse width f1 and amplitude f2 of the second reference pulse wave signals using a preset program to generate second reference biometric information such as blood pressure, electrocardiogram, heart rate, etc. For example, characteristics such as the pulse width f1 and the amplitude f2 of the second reference pulse wave signals may be detected differently from characteristics such as the pulse width f1 and the amplitude f2 of the first reference pulse wave signals.

The biometric information detection device transmits the second reference pulse wave signals and the second reference biometric information to a wireless communication module such as Bluetooth and Wi-Fi, and the main driving circuit 200 receives the second reference pulse wave signals and the second reference biometric information through the wireless communication module and the short-range communication unit 211. In some cases, the biometric information detection device directly transmits the second reference pulse wave signals and the second reference biometric information to the short-range communication unit 211

FIG. 14 is an example of a waveform diagram illustrating a detection waveform of a pulse wave signal detected through a display device of FIG. 12. For example, the y-axis of the waveform diagram represents PPG signal and the x-axis of the waveform diagram represents time.

Referring to FIG. 14, the main processing unit of the main driving circuit 200 or the biometric signal output unit 224 of the main driving circuit 200 guides the pulse wave signal detection process of the left hand through the application program screen. Accordingly, the user may bring the left hand into contact with the display screen displayed with the application program screen, where the display device 10 is able to measure second pulse wave signals PG2_S. The second pulse wave signal input unit 222 receives light sensing signals through the light sensing lines ERL of the display panel 100 and transmits the second pulse wave signals PG2_S based on the light sensing signals to the second reference correction value setting unit 213.

The second reference correction value setting unit 213 substitutes the numerical characteristic information of the second pulse wave signals PG2_S based on the numerical characteristic information of the second reference pulse wave signals, and generates second reference correction values based on at least one calculation result among the median value, the average value, and the difference value between the numerical characteristic information of the second pulse wave signals PG1_S and the numerical characteristic information of the second reference pulse wave signals.

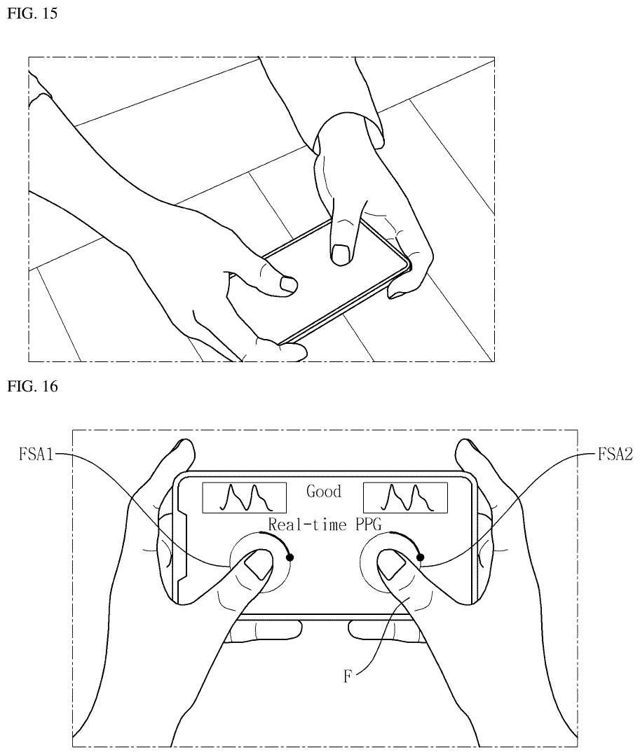

FIG. 15 is an example of a diagram illustrating a measuring process of a plurality of pulse wave signals and biometric information using both thumbs. FIG. 16 is an example of a diagram illustrating a displayed image display screen when a pulse wave signal is detected during a measurement period.

Referring to FIGS. 15 and 16, the main driving circuit 200 guides a pulse wave signal detection process with the application program screen so that the first and second pulse wave signals PG1_S and PG2_S may be detected through both fingers of the left and right hands when measuring the biometric information of the.

During the period of detecting biometric information, the main driving circuit 200 supplies data voltage to the first and second unit pixels PG1 and PG2 of the display panel 100. In some cases, the main driving circuit 200 supplies control signals to the display scan driver 110 and the light sensing scan driver 120 to display a preset application program screen for detecting biometric signals, for example, pulse wave signals, in the display area DA.

The main driving circuit 200 displays a plurality of biometric information measurement areas (e.g., a first biometric information measurement area FSA1 and a second biometric information measurement area FSA2) that guide the touch positions of body parts such as a finger F can be placed on a guide screen of the application program. In some cases, a wave form of the biometric signal detected in real time, that is, the pulse wave signals PG1_S and PG2_S is displayed in a display window to guide the overall pulse wave signal detection process. In some cases, for example, the main driving circuit 200 displays the period required for measuring biometric information, the pulse wave signal detection period, and the first and second biometric information measurement areas FSA1 and FSA2 in a circular or bar graph form through the application program screen.

For example, through the touch driving circuit 400, the main driving circuit 200 receives a plurality of touch position coordinates sensed through a touch sensing unit TSU or a pressure sensing unit PSU. In some cases, the main driving circuit 200 supplies the data voltage to the first and second unit pixels PG1 and PG2 aligned respectively in the plurality of biometric information measurement areas FSA1 and FSA2 and supplies control signals to the display scan driver 110 and the light sensing scan driver 120. In some cases, the main driving circuit 200 may supply a preset data voltage to at least one display pixel among first and second display pixels SP1 and SP2 of the first and second unit pixels PG1 and PG2. Accordingly, an optical signal may be detected by at least one light of the green light and the red light. Then, the main driving circuit 200 receives an optical signal, for example, the light sensing signals, from light sensing pixels LSP aligned respectively in the plurality of biometric information measurement areas FSA1 and FSA2 through the light sensing lines ERL.

The first and second pulse wave signal input units 221 and 222 of the main driving circuit 200 detect the first and second pulse wave signals PG1_S and PG2_S respectively corresponding to light sensing signals for each biometric information measurement areas FSA1 and FSA2 that are received in real time. In some cases, the first and second pulse wave signals PG1_S and PG2_S as digital signal data. Each of the pulse wave signals are a signal corresponding to the magnitude and magnitude change of the light sensing signals. The main driving circuit 200 displays the first and second pulse wave signals PG1_S and PG2_S detected in real time in graphic form of a graph type in a display window of the application program screen.

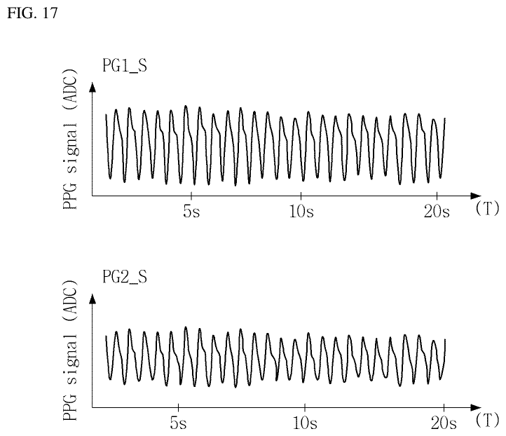

FIG. 17 is an example of a waveform diagram illustrating pulse wave signals detected from different touch positions. Referring to FIG. 17, the programming processor 223 of the main driving circuit 200 receives the first and second pulse wave signals PG1_S and PG2_S in real time through the first and second pulse wave signal input units 221 and 222. Characteristics such as a pulse width and amplitude of the first and second pulse wave signals PG1_S and PG2_S may be detected to be different from each other. In some cases, the first pulse wave signal PG1_S corresponds to the pulse wave signal based on the right finger, and the second pulse wave signal PG2_S corresponds to the pulse wave signal based on the left finger, or vice versa. In some cases, the waveform graph represents the PPG signal as a function of time.

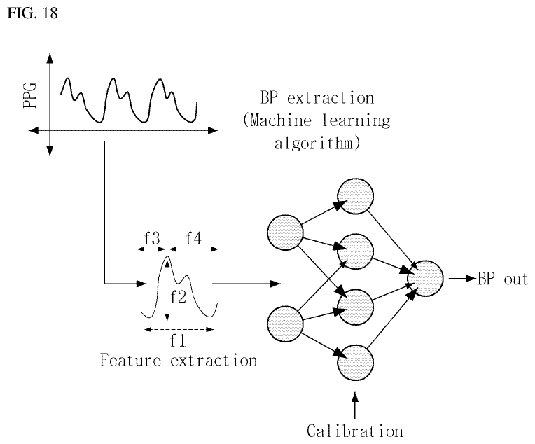

FIG. 18 is an example of a diagram illustrating a method for calculating blood pressure information using a machine learning algorithm according to an embodiments of the present inventive concept. Referring to FIG. 18, the programming processor 223 of the main driving circuit 200 analyzes the first and second pulse wave signals PG1_S and PG2_S inputted in real time, respectively, based on the prestored first and second reference correction values using a preset machine learning algorithm.

For example, the programming processor 223 compares characteristics change information of the first pulse wave signals PG1_S with the characteristics information of the first reference correction values and detects the comparison result. Then, the comparison results are converted into a database as training data. For example, the programming processor 223 may compare the characteristics information such as the pulse width f1 (e.g., systolic and diastolic cycles) and the numeric information of the first reference correction values, the systolic cycle amplitude f2 (e.g., systolic blood pressure values) of the first reference correction values, the change information of the systolic pulse width f3 (e.g., systolic cycle) of the first reference correction values, and the change information of the diastolic pulse width f4 (e.g., diastolic cycle) of the first reference correction values with the characteristic information of the first pulse wave signals PG1_S. For example, the characteristic information of the first pulse wave signals PG1_S include the pulse width f1 (e.g., systolic and diastolic cycles) and the number information, the amplitude f2 (e.g., systolic blood pressure values) of the systolic cycle, the change information of the systolic pulse width f3 (e.g., systolic cycle), and the change information of the diastolic pulse width f4 (e.g., diastolic cycle) to detect the comparison results. In some cases, the comparison results are stored in a database as training data.

In some embodiments, the programming processor 223 may use a machine learning algorithm to calculate the first biometric information with respect to a blood pressure, a heart rate, heart rate variability, a respiratory rate, blood vessel elasticity, a cardiovascular disease analysis result, and oxygen saturation based on the change magnitude or change rate of the characteristics information of the first pulse wave signals PG1_S in comparison with the characteristics information of the first reference correction values. In some cases, the programming processor 223 may use a machine learning algorithm to analyze each of a high pulse cycle and a high pulse cycle change of the first pulse wave signals PG1_S, a high pulse magnitude and a change thereof, a low pulse magnitude and a change thereof, a waveform change of the high pulse, a period in which the high pulse reaches a peak, and a difference in pulse magnitude between first pulse wave signals PG1_S respectively detected by green light and red light in comparison with the characteristics information of the first reference correction values. Then, the first biometric information with respect to a blood pressure, a heart rate, heart rate variability, a respiratory rate, blood vessel elasticity, a cardiovascular disease analysis result, and oxygen saturation based on each analysis result are obtained.

In some embodiments, the programming processor 223 may estimate blood pressures of finger blood vessels based on time differences between the time points corresponding to the peaks of the first pulse wave signals PG1_S and the time points corresponding to the peaks of the filtered pulse waves. For example, pulse wave signals for a preset period before and after the time points corresponding to the peaks of the first pulse wave signals PG1_S may be calculated, and thus the differences in the pulse wave signals may represents the biometric information of the blood pressure. Among the estimated blood pressures, the blood pressure with the maximum magnitude may be calculated as systolic blood pressure, and the blood pressure with the minimum magnitude may be calculated as diastolic blood pressure. In some cases, other blood pressure such as an average blood pressure may be calculated using the estimated blood pressures.

Similarly, the programming processor 223 may use a machine learning algorithm to calculate the second biometric information with respect to a blood pressure, a heart rate, heart rate variability, a respiratory rate, blood vessel elasticity, a cardiovascular disease analysis result, and oxygen saturation based on the change in magnitude or rate of the characteristic information of the second pulse wave signals PG2_S in comparison with the characteristics information of the second reference correction values.

Thereafter, the biometric signal output unit 224 selects one of biometric information with an abnormal level (e.g., high blood pressure level) among the first and second biometric information measured by the programming processor 223 as the real-time biometric information of the user and displays the selected biometric information on the application screen. For example, the biometric signal output unit 224 may generate digital video data corresponding to touch coordinates based on the touch coordinate data, or may execute an application indicated by an icon displayed on the touch coordinates of the user.

In some cases, the machine learning algorithm is performed using a machine learning model. For example, the machine learning model is designed to recognize patterns, make predictions, or perform a specific task (for example, generate biometric information) without being explicitly programmed. In some cases, the machine learning model includes a plurality of artificial neural networks (ANNs) trained to generate a predicted biometric information based on an input signal. Further detail on the machine learning model is described with reference to FIG. 12.

In some cases, a first neural network layer of the machine learning model is trained to receive input data (e.g., the extracted features from a waveform diagram in FIG. 17). In some cases, the extracted features include the pulse width f1, the systolic cycle amplitude f2, the change information of the systolic pulse width f3, and the change information of the diastolic pulse width f4. In some cases, each of the extracted features may be represented as a vector.

Then, an output of the first neural network layer is provided to an intermediate neural network layer(s) of the machine learning model for analysis and computations. Then, an output of the intermediate neural network layer is provided to the final neural network layer of the machine learning model to generate the predicted biometric information (e.g., the output blood pressure).

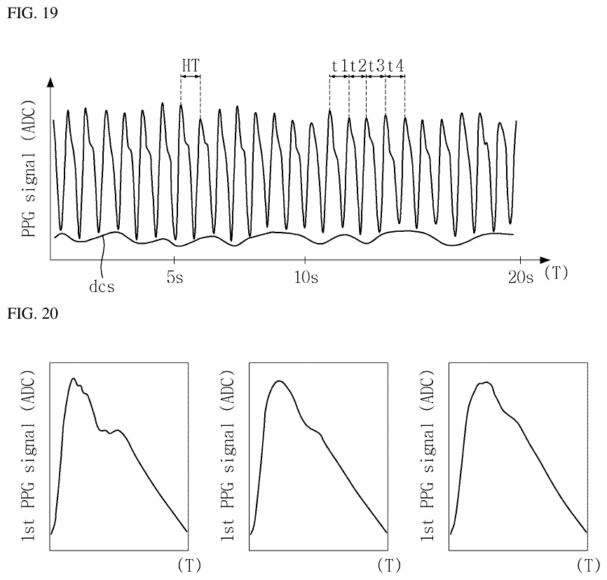

FIG. 19 is an example of a graph illustrating a method for calculating information on a heart rate and respiration among biometric information according to an embodiment. For example, the y-axis of the graph represents PPG signal and the x-axis of the graph represents the time.

Referring to FIG. 19, the main driving circuit 200 samples first pulse wave signals PG1_S during a preset sampling period before and after the time points corresponding to the peaks of the first pulse wave signals PG1_S, and detects a generation period HT of high pulses with respect to the sampled first pulse wave signals PG1_S. In some cases, biometric information with respect to the heart rate cycle and heart rate HR may be detected by computing the number of occurrences of high pulses for each preset reference period (e.g., 60 seconds) with respect to the sampled first pulse wave signals PG1_S.

For example, the main driving circuit 200 detects the heart rate cycle HT and the heart rate cycle changes t1 to t4 of high pulses for each preset reference period for the peaks of the pulse wave signal to detect the heart rate variability HRV based on the heart rate cycle change rate.

In some cases, the main driving circuit 200 sequentially detects the generation cycle and the magnitude value of low pulses of the sampled first pulse wave signals PG1_S. Then, the change cycle of a magnitude value des of low pulses may be detected in units of preset reference periods (for example, 60 seconds) to detect the respiratory change state and the respiratory rate RR of a user. For example, the cycle in which the magnitude value des of low pulses increases and the cycle in which the magnitude value des of low pulses decreases may be analyzed to detect the respiratory change state and the respiratory rate RR of the user using the increasing cycle and the decreasing cycle of the magnitude value des of low pulses.

FIG. 20 is an example of a graph illustrating a method for calculating information on blood vessel elasticity among biometric information according to an embodiment. for example the graph represents the PPG signal as a function of time. Referring to FIG. 20, the main driving circuit 200 may set and obtain the blood vessel elasticity BVE by expanding and analyzing the high pulse variation of the sampled first pulse wave signals PG1_S.

When the blood flow increases due to heartbeat, the pulse wave signal is changed to a high pulse form. In some cases, when the blood flow decreases, the pulse wave signal is changed to a low pulse form. If the blood flow changes rapidly due to the shape of the blood vessel during the period in which the blood flow increases or decreases, the change in the blood flow may be quickly relaxed or slowed based on the elasticity of the blood vessel. Accordingly, the main driving circuit 200 sets and obtains the BVE using a value corresponding to the magnitude of changes in high pulses by expanding and analyzing the high pulse change form of the first pulse wave signals PG1_S.

FIG. 21 is an example of a graph illustrating a method for calculating information on a cardiovascular disease among biometric information according to an embodiment. In some cases, the y-axis of the graph may represent the PPG signal and the x-axis of the graph may represent the time.

Referring to FIG. 21, the main driving circuit 200 may set and obtain a cardiovascular disease evaluation score (or a cardiovascular health analysis result score) by differentiating, expanding, and analyzing the high pulse change form of the sampled first pulse wave signals PG1_S. For example, the main driving circuit 200 detects a period (Crest Time) in which the first pulse wave signals PG1_S reach the peak PK in a high pulse form, and time variation ΔT in which the first pulse wave signals PG1_S fall compared to the period (Crest Time) in which the first pulse wave signals PG1_S reach the peak PK. As the period (Crest Time) in which the first pulse wave signals PG1_S reach the peak PK in a high pulse form increases, the risk of heart disease increases. Accordingly, the main driving circuit 200 may set and obtain the cardiovascular disease evaluation score (or the cardiovascular health analysis result score) in inverse proportion to the period (Crest Time) in which the first pulse wave signals PG1_S reach the peak PK in a high pulse form.

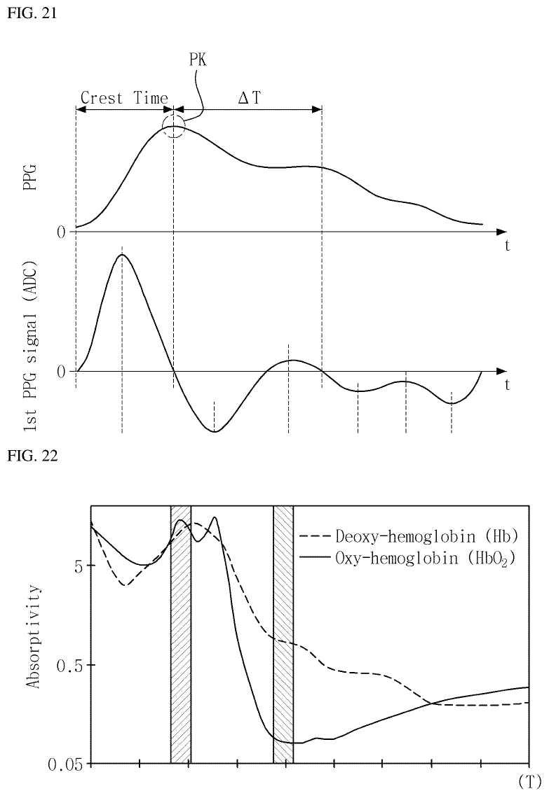

FIG. 22 is an example of a graph illustrating a method for calculating information on oxygen saturation among biometric information according to an embodiment. In some cases, the graph represents the absorptivity as a function of time.

Referring to FIG. 22, when the heart contracts, red blood cells carry more oxygen hemoglobin to peripheral tissues. On the other hand, when the heart relaxes, the heart receives a partial influx of blood from the peripheral tissues. Thus, the main driving circuit 200 detects a deoxy-hemoglobin (Hb) value using the magnitude change of the first pulse wave signals PG1_S detected by the green light, and detects a HbO₂ (Oxy-hemoglobin) value using the magnitude change of the first pulse wave signals PG1_S detected by the red light.

The main driving circuit 200 may detect the oxygen saturation (SpO₂) using the following Eq. (2).

$${Sp O}_{2} = {Hb O}_{2} / {Sp O}_{2} + Hb \qquad ( 2 )$$

where HbO₂ represents the oxy-hemoglobin and Hb represents the deoxy-hemoglobin.

Accordingly, the main driving circuit 200 may display the biometric information such as the blood pressure BP, the heart rate HR, the heart rate variability HRV, the respiratory rate RR, the blood vessel elasticity BVE, the cardiovascular disease (or the cardiovascular health analysis result score), the oxygen saturation (SpO₂), or the like on the application program screen.

Although embodiments of the present disclosure have been described with reference to the accompanying drawings, the present disclosure is not necessarily limited to the above embodiments. For example, those skilled in the art will appreciate that many variations and modifications can be made to the preferred embodiments without substantially departing from the principles of the present disclosure. Therefore, the disclosed preferred embodiments of the disclosure are used in a generic and descriptive sense and not for purposes of limitation.

What is claimed is:

1. A display device comprising:
a display panel including display pixels and light sensing pixels arranged in a display area of the display panel;
a display scan driver configured to drive the display pixels to emit light;
a light sensing scan driver configured to drive the light sensing pixels to sense light; and
a main driving circuit configured to perform operations comprising:
obtaining, using a biometric information detection device, a first reference pulse wave signal, a second reference pulse wave signal, a first pulse wave signal, and a second pulse wave signal;
generating a first reference correction value and a second reference correction value based on the first reference pulse wave signal and the second reference pulse wave signal, respectively; and
generating biometric information based on the first pulse wave signal, the second pulse wave signal, the first reference correction value, and the second reference correction value.

2. The display device of claim 1, wherein the main driving circuit is further configured to perform operations comprising:
obtaining the first reference pulse wave signal through a left arm and the first pulse wave signal through a right finger;
obtaining the second reference pulse wave signal through a right arm and the second pulse wave signal through a left finger;
generating the first reference correction value with respect to the right finger based on an average value or a difference value between the first pulse wave signal and the first reference pulse wave signal; and
generating the second reference correction value with respect to the left finger based on an average value or a difference value between the second pulse wave signal and the second reference pulse wave signal.

3. The display device of claim 2, wherein the main driving circuit is further configured to perform operations comprising:
guiding a detection process of the first pulse wave signal and the second pulse wave signal using an application program screen;
generating, using a machine learning algorithm, a first biometric information based on the first pulse wave signal and the first reference correction value;
generating, using the machine learning algorithm, a second biometric information based on the second pulse wave signal and the second reference correction value;
generating a final measuring result based on the first biometric information and the second biometric information; and
displaying the final measuring result on the application program screen.

4. The display device of claim 3, wherein the main driving circuit is further configured to perform operations comprising:
computing a first numerical characteristic information based on the first reference pulse wave signal;
computing a second numerical characteristic information based on the second reference pulse wave signal;
generating the first reference correction value based on an average value or a difference value between the first numerical characteristic information and a numerical characteristic information of the first pulse wave signal; and
generating the second reference correction value based on an average value, a difference value, or a median value between the second numerical characteristic information and a numerical characteristic information of the second pulse wave signal.

5. The display device of claim 3, wherein the main driving circuit is further configured to perform operations comprising:
obtaining a third reference pulse wave signal and a third pulse wave signal;
generating a first average reference correction value based on the first reference pulse wave signal, the first pulse wave signal, the third reference pulse wave signal, and the third pulse wave signal, wherein the first reference correction value is generated based on the first average reference correction value;
obtaining a fourth reference pulse wave signal and a fourth pulse wave signal;
generating a second average reference correction value based on the second reference pulse wave signal, the second pulse wave signal, the fourth reference pulse wave signal, and the fourth pulse wave signal, wherein the second reference correction value is generated based on the second average reference correction value.

6. The display device of claim 2, wherein the main driving circuit further comprises:
a short-range communication unit configured to receive the first reference pulse wave signal and the second reference pulse wave signal;
a first pulse wave signal input unit configured to receive the first pulse wave signal of the right hand;
a second pulse wave signal input unit configured to receive the second pulse wave signal of the left hand;
a first reference correction value setting unit configured to generate the first reference correction value;
a second reference correction value setting unit configured to generate the second reference correction value; and
a reference correction value storing unit configured to store the first reference correction value and the second reference correction value.

7. The display device of claim 6, wherein:
the first reference correction value setting unit generates a first numerical characteristic information based on the first reference pulse wave signal, wherein the first numerical characteristic information includes an amplitude and a pulse width range, a systolic cycle and a diastolic cycle, a systolic blood pressure change width range, a systolic cycle, and a diastolic cycle change range; and
wherein the first reference correction value setting unit generates the first reference correction value based on an average value, a difference value, or a median value between a numerical characteristic information of the first pulse wave signal and the first numerical characteristic information.

8. The display device of claim 6, wherein:
the second reference correction value setting unit generates a second numerical characteristic information based on the second reference pulse wave signal, wherein the second numerical characteristic information includes an amplitude and a pulse width range, a systolic cycle and diastolic cycle, a systolic blood pressure change width range, a systolic cycle, and a diastolic cycle change range; and
wherein the second reference correction value setting unit generates the second reference correction value based on an average value, a difference value, or a median value between a numerical characteristic information of the second pulse wave signal and the second numerical characteristic information.

9. The display device of claim 6, wherein the main driving circuit further comprises:
a programming processor configured to generate first biometric information and second biometric information based on the first pulse wave signal and the second pulse wave signal, respectively in real time during a measurement period, and configured to analyze the first biometric information and the second biometric information based on the first reference correction value and the second reference correction value using a preset machine learning algorithm; and
a biometric signal output unit configured to generate a final measuring result based on the first biometric information and the second biometric information, and configured to display the final measuring result on the application program screen.

10. The display device of claim 9, wherein:
the programming processor is configured to generate the first biometric information by analyzing the first pulse wave signal based on the first reference correction value using a machine learning algorithm, and
wherein the biometric signal output unit is configured to generate the final measuring result based on the first biometric information and the second biometric information.

11. A display device comprising:
a display panel including display pixels and light sensing pixels arranged in a display area of the display panel;
a display scan driver configured to drive the display pixels to emit light;
a light sensing scan driver configured to drive the light sensing pixels to sense light; and
a main driving circuit configured to perform operations comprising:
obtaining, using a biometric information detection device, a first reference pulse wave signal, a second reference pulse wave, a first pulse wave signal, and a second pulse wave signal;
generating a first reference correction value and a second reference correction value based on the first reference pulse wave signal and the second reference pulse wave signal; and
generating biometric information based on the first pulse wave signal, the second pulse wave signal, the first reference correction value, and the second reference correction value,
wherein the main driving circuit guides a biometric signal detection process by displaying an application program screen, generating a biometric signal based on the first pulse wave signal and second pulse wave signal, generating a final measuring result based on the biometric signal, and displaying the final measuring result on the application program screen.

12. The display device of claim 11, wherein the main driving circuit is further configured to perform operations comprising:
obtaining the first reference pulse wave signal through a left arm and the first pulse wave signal through a right finger;
obtaining the second reference pulse wave signal through a right arm and the second pulse wave signal through a left finger;
generating the first reference correction value with respect to the right finger based on an average value or a difference value between a numerical characteristic information of the first pulse wave signal and a numerical characteristic information of the first reference pulse wave signal; and
generating the second reference correction value with respect to the left finger based on an average value or a difference value between a numerical characteristic information of the second pulse wave signal and a numerical characteristic information of the second reference pulse wave signal.

13. The display device of claim 12, wherein the main driving circuit is further configured to perform operations comprising:
guiding a detection process of the first pulse wave signal and the second pulse wave signal using an application program screen;
generating, using a machine learning algorithm, a first biometric information based on the first pulse wave signal and the first reference correction value;
generating, using a machine learning algorithm, a second biometric information based on the second pulse wave signal and the second reference correction value;
generating a final measuring result based on the first biometric information and the second biometric information; and
displaying the final measuring result on the application program screen.

14. The display device of claim 13, wherein the main driving circuit is further configured to perform operations comprising:
computing a first numerical characteristic information based on the first reference pulse wave signal;
computing a second numerical characteristic information based on the second reference pulse wave signal;
generating the first reference correction value based on an average value or a difference value between the first numerical characteristic information and a numerical characteristic information of the first pulse wave signal; and
generating the second reference correction value based on an average value, a difference value, or a median value between the second numerical characteristic information and a numerical characteristic information of the second pulse wave signal.

15. The display device of claim 12, wherein the main driving circuit comprises:
a short-range communication unit configured to receive the first reference pulse wave signal and the second reference pulse wave signal;
a first pulse wave signal input unit configured to receive the first pulse wave signal of the right hand;
a second pulse wave signal input unit configured to receive the second pulse wave signal of the left hand;
a first reference correction value setting unit configured to generate the first reference correction value;
a second reference correction value setting unit configured to generate the second reference correction value; and
a reference correction value storing unit configured to store the first reference correction value and the second reference correction value.

16. The display device of claim 15, wherein:
the first reference correction value setting unit generates a first numerical characteristic information based on the first reference pulse wave signal, wherein the first numerical characteristic information includes an amplitude and a pulse width range, a systolic cycle and a diastolic cycle, a systolic blood pressure change width range, a systolic cycle, and a diastolic cycle change range; and
wherein the first reference correction value setting unit generates the first reference correction value based on an average value, a difference value, or a median value between a numerical characteristic information of the first pulse wave signal and a first numerical characteristic information.

17. The display device of claim 15, wherein:
the second reference correction value setting unit generates a second numerical characteristic information based on the second reference pulse wave signal, wherein the second numerical characteristic information includes an amplitude and a pulse width range, a systolic cycle and a diastolic cycle, a systolic blood pressure change width range, a systolic cycle, and a diastolic cycle change range; and
wherein the second reference correction value setting unit generates the second reference correction value based on an average value, a difference value, a the median value between a numerical characteristic information of the second pulse wave signal and the second numerical characteristic information.

18. The display device of claim 15, wherein the main driving circuit further comprises:
a programming processor configured to generate first biometric information and second biometric information based on the first pulse wave signal and the second pulse wave signal, respectively, in real time during a measurement period, and configured to analyze the first biometric information and the second biometric information based on the first reference correction value and second reference correction value using a preset machine learning algorithm; and
a biometric signal output unit configured to generate a final measuring result based on the first biometric information and the second biometric information, and configured to display the final measuring result on the application program screen.

19. A method for generating biometric information using a display device, the method comprising:
obtaining, using a biometric information detection device, a first reference pulse wave signal through a left arm;
obtaining, using a display device, a first pulse wave signal through a right finger;
generating a first reference correction value with respect to the right finger based on the first reference pulse wave signal and the first pulse wave signal; and
generating biometric information based on the first reference correction value.

20. The method of claim 19, further comprising:
obtaining, using the biometric information detection device, a second reference pulse wave signal through a right arm;
obtaining, using the display device, a second pulse wave signal through a left finger;
generating a second reference correction value with respect to the left finger based on the second reference pulse wave signal and the second pulse wave signal; and
generating an additional biometric information based on the second reference correction value, wherein the biometric information is generated based on the additional biometric information.
